(19) United States
(12) Patent Application Publication
(10) Pub. No.: US 2015/0128305 A1
(43) Pub. Date: May 7, 2015

(54) POLYNUCLEOTIDE ENCODING CaTLP1 PROTEIN AND USES THEREOF

(71) Applicant: National Institute of Plant Genome Research, New Delhi (IN)

(72) Inventors: Niranjan Chakraborty, New Delhi (IN); Subhra Chakraborty, New Delhi (IN); Asis Datta, New Delhi (IN); Vijay Wardhan, New Delhi (IN); Kishwer Jahan, New Delhi (IN)

(21) Appl. No.: 14/399,706

(22) PCT Filed: May 7, 2013
(86) PCT No.: PCT/IN2013/000302

§ 371 Date: Nov. 7, 2014

(30) Foreign Application Priority Data
May 7, 2012 (IN) 1406/DEL/2012

(51) Int. Cl. C12N 15/82; C07K 14/415

(52) U.S. Cl. CPC C12N 15/8273; C07K 14/415; C12N 15/8271
USPC 800/293; 536/23.6; 435/320.1; 435/252.33; 435/252.2; 435/254.2; 435/419; 530/370; 435/468; 800/278; 800/294; 800/298; 800/317.3; 800/317.2; 800/306; 800/317.4; 800/309; 800/322; 800/317.1; 800/320; 800/314; 800/312; 800/323.1; 800/320.2; 800/320.1; 800/320.3; 800/323

(57) ABSTRACT

The present invention provides herein a polynucleotide sequence encoding the tubby-like protein, CaTLP1, from chickpea (Cicer arientium L.) that is responsive to abiotic stress and is involved in plant growth and development. Further, the recombinant DNA construct and recombinant vector comprising the polynucleotide sequence encoding CaTLP1, host cell comprising the recombinant vector and a process for producing a transgenic plant that expresses the CaTLP1 protein are also provided herein.

### FIELD OF INVENTION

The present invention relates to the field of plant molecular biology, genetic engineering, and transgenic plant, in particular to stress responsive protein, CaTLP1, from Cicer arietinum L., and its uses thereof. The invention further relates to a method for producing modified plants that exhibit enhanced tolerance to abiotic stress, and the modified plants produced by the process.

### BACKGROUND OF THE INVENTION

Plant environmental (abiotic) stress has been defined as “any change in environmental conditions that might reduce or adversely change a plant's growth or development” (J. Levitt, 1972, Responses of Plants to Environmental Stresses, Academic Press Inc., New York and London). Adverse environmental factors such as high winds, cold (chilling and freezing), heat, desiccation (drought), flood, salinity, soil mineral deficiency, soil mineral toxicity, and other unfavourable growing conditions are common stresses that affect environments constantly. Prolonged exposure to abiotic stresses can result in severe crop damage and impacts the growth and productivity of crops worldwide. Continuous exposure to abiotic stresses results in alterations in the plant metabolism which ultimately leads to cell death and consequently yield losses. Crop losses and crop yield losses of major crops such as rice, wheat, and maize caused by environmental stresses represent a significant economic and political factor that contribute to food shortage in many underdeveloped countries.

Environmental stress limits growth, development, and productivity of the crops, thereby playing a major role in determining the geographic distribution of many plant species. Major environmental stresses are united by the fact that at least a part of their detrimental effect on plant performance is caused by disruption of water status.

Water-deficit or dehydration is the most common environmental stress to which plants are often exposed, and in many regions forms the bottleneck to agricultural development. Dehydration is the most crucial environmental constraint on plant growth and development and agricultural productivity. Transitory or constant dehydration adversely affects the morpho-anatomical, physiological, and biochemical processes in plants, though the extent of damage varies considerably at different developmental stages. The perception and dehydration-induced signal transduction to switch on adaptive responses are critical steps in determining the survival of plants exposed to dehydration, and elucidation of the nature of these mechanisms has been an interesting area of research. The plant cell wall or extracellular matrix (ECM) is a dynamic organization essential not only for cell division, enlargement, and differentiation; but also acting in diverse environmental stress. Though the protein part accounts for only 10% of the cell wall mass, the proteins contribute substantially to the cellular dynamics as they comprise several hundreds of different molecules with diverse functions.

Plants are typically exposed to conditions of reduced environmental water content in different stages of their life cycle. Most plants have evolved strategies and specific patterns of stress mediated metabolism to protect themselves against the conditions of low water or desiccation. Developing stress-tolerant plants is a strategy that has the potential to solve or mediate at least some of the problems arising due to stress response in the plant. However, traditional plant breeding strategies to develop new lines of plants that exhibit resistance (tolerance) to stresses are relatively slow and require specific resistant lines for crossing with the desired line. Most of the efforts to mitigate the effects of plant stress have included complex methodologies that are both time consuming and expensive. Limited germplasm resources for stress tolerance and incompatibility in crosses between distantly related plant species represent the significant problems encountered in conventional breeding. Additionally, the cellular processes leading to drought, cold, and salt tolerance are complex in nature and involve multiple mechanisms of cellular adaptation and numerous metabolic pathways which makes the development of mitigation strategies increasingly difficult. The development of resistance to abiotic stress has previously been demonstrated by the use of mitigating compounds and isolated polynucleotide sequences.

U.S. Pat. No. 6,534,446 discloses methods for mitigating the effects of plant stress by use of plant stress mitigating compounds and compositions comprising gamma immunobutyric acid and glutamic acid.

U.S. Pat. No. 8,071,843 discloses a method for increasing stress resistance in plants by introducing an isolated polynucleotide encoding a raffinose synthase into the body of a plant, and selecting transformed plants having raffinose synthetic activity in the plant body greater than the wild type plant grown under the same conditions.

U.S. Pat. No. 7,897,843 discloses plant transcription factor polypeptides, polynucleotides that encode them, homologs from a variety of plant species, and methods of using the polynucleotides and polypeptides to produce transgenic plants having advantageous properties, including increased biomass or improved cold or other osmotic stress tolerance, as compared to wild-type or reference plants.

U.S. Pat. No. 7,368,632 discloses transgenic plants comprising a plant gene (ROBS), demonstrating increased plant growth, plant vigor, and improvement in the capacity to tolerate a variety of stress conditions.

Plants harbour a large number of tubby-like proteins (TLPs); for instance, 11 members in Arabidopsis (Lai C-P, Lee C-L, Chen P-H, Wu S-H, Yang C-C, Shaw J-F; 2004, Molecular analysis of the Arabidopsis TUBBY-like protein gene family; Plant Physiology; 134: 1586-1597), and 14 members in rice (Liu Q, 2008, Identification of rice TUBBY-like genes and their evolution. FEBS J 275: 163-171; Kou Y, Qiu D, Wang L, Li X, Wang S, 2009, Molecular analyses of the rice tubby-like protein gene family and their response to bacterial infection, Plant Cell Reports, 28: 113-121). In addition to the typical 270 amino acids conserved C-terminal Tub domain, plant TLPs have evolved with an F-box conserved at N-terminal sequence, which is otherwise highly divergent in animal. It has also been shown that these proteins may function as bipartite transcription regulators by binding to double-stranded DNA and activating transcription (Boggon T J, Shan W S, Santagata S, Myers S C, Shapiro L, 1999, Implication of tubby proteins as transcription factors by structure-based functional analysis, Science, 286: 2119-2125).

While a wide array of cellular functions of TLPs have been established in animals, their role in plants is still elusive. Nevertheless, the highly conserved evolution of tubby proteins and the existence of redundancy suggest their indispensable role in plants. In recent years; this protein family has been shown to be involved in abscisic acid (ABA)-dependent signalling in Arabidopsis and pathostress response in rice. The mode of action of tubby family proteins in plants is believed to be regulated by ABA, which is characteristically implicated in different stress responses.

The multi-component nature of stress tolerance has not only made breeding for tolerance largely unsuccessful, but has also limited the ability to genetically engineer stress tolerance plants using biotechnological methods. Thus, there is a need for novel stress resistant or tolerant genes, such that better adaptation may be sought in plants leading to a healthy and robust agronomy.

### SUMMARY OF THE INVENTION

Accordingly it is an aspect of the present invention to provide a polynucleotide encoding the protein having the amino acid sequence as set forth in SEQ ID NO: 5.

It is another aspect of the present invention to provide a polypeptide having amino acid sequence as set forth in SEQ ID NO: 5, wherein expression of the polypeptide is responsible for conferring enhanced tolerance to abiotic stress in a transformed plant as compared to an untransformed plant.

It is yet another aspect of the present invention to provide a method for producing a transformed plant cell, plant, or a part thereof over-expressing a protein having the amino acid sequence as set forth in SEQ ID NO: 5, wherein the method comprises: transforming the plant cell, plant, or part thereof with a polynucleotide encoding the protein having the amino acid sequence as set forth in SEQ ID NO: 5, and selecting the transformed plant cell, plant or part thereof, wherein the transformed plant cell, plant, or part thereof is more tolerant to abiotic stress than an untransformed plant.

It is still another aspect of the present invention to provide a transgenic plant, plant cell or progeny thereof over-expressing a protein having the amino acid sequence as set forth in SEQ ID NO: 5, wherein the transgenic plant, plant cell or progeny thereof is more tolerant to abiotic stress than an untransformed plant.

### BRIEF DESCRIPTION OF THE ACCOMPANYING DRAWINGS

The following drawings form part of the present specification and are included to further illustrate aspects of the present invention. The invention may be better understood by reference to the drawings in combination with the detailed description of the specific embodiments presented herein.

FIG. 1(a) shows the results of electrofocusing of cell wall proteins on IPG strip. FIG. 1(b) shows the results of electrofocusing denoting temporal changes of TLP under progressive dehydration and recovery stage at 24 hours (R24). The bar graphs show the graphical representation the fold-expression in terms of band intensity of TLP plotted against the duration of dehydration.

FIG. 2 shows expression profile of CaTLP1 in response to different stresses and phytohormone treatments. Three-week-old chickpea seedlings were subjected to (a) dehydration, (b) ABA (100 μM) treatment, (c) salt stress at different concentration of NaCl (100-500 mM), (d) methyl jasmonate (100 μM), and salicylic acid (5 mM). The lanes represent various time points during the treatments. (e) Northern blot showing differential expression of CaTLP1 in different organs with 10 μg total RNA in each lane. The RNA blot was hybridized with a [³²P]-labelled 0.5 kb 5′-CaTLP1 fragment. The image of ethidium bromide-stained rRNA shows equivalent loading and RNA quality. R, root; S, stem; L, leaf; F, flower; SP, seed pod.

FIG. 3 shows copy number of CaTLP1. Genomic DNA (10 μg) of chickpea seedlings was digested with restriction endonucleases as indicated, and Southern blot was hybridized with [³²P]-labelled 314-bp fragment of CaTLP1. Molecular weight marker in kb is indicated on the left.

FIG. 4 shows the results of structural and phylogenetic analysis of CaTLP1. FIG. 2(a) shows a schematic representation of the CaTLP1 and exon-intron organization and FIG. 2(b) shows the results for an unrooted phylogenetic tree showing evolutionary relationship of CaTLP1 with its orthologs.

FIG. 5 shows isolation and purification of recombinant GST-CaTLP1 protein expressed in E. coli (a) Time course induction of GST-CaTLP1. Induced expression of proteins is indicated by arrows (b) Affinity purification of GST-CaTLP1 recombinant protein Lane1 represents purified GST proteins while lanes 2, 3 and 4 represent samples from crude bacterial extract, GST bead binding and purified protein, respectively. Arrow represents the expressed protein.

FIG. 6 shows ESI-MS analysis of CaTLP1 showing “multicharge envelope” of signals from differentially charged forms of the protein.

FIG. 7 shows (a) CaTLP1 demonstrates a preference for binding double-stranded DNA. Gel-shift assays were carried out using double-stranded 20 mer d(A/T) and d(G/C), and (b) single-stranded 20 mer d(C₂₀) and d(T₂₀) [³²P]-labelled probes. The shift was competed out by 100-fold excess of cold oligos. The arrows represent the band-shifts. (c) Transactivation analysis of CaTLP1. The CaTLP1 coding sequence fused with GAL4-DB in the vector pGBKT7 was used for transformation into a yeast strain AH109. Yeast colonies harbouring no vector, empty vector (pGBKT7), and recombinant pGBKT7-CaTLP1 were grown on YPDA, synthetic medium lacking Trp, and on SD media lacking Trp but supplemented with X-α-gal. The GAL4AD domain from pGADT7 was cloned in the vector pGBKT7 to be used as positive control. FP, free probe.

FIG. 8 shows Subcellular localization of CaTLP1-YFP fusion protein. The roots of stably transformed A. thaliana show the expression of CaTLP1-YFP. (a) Confocal images of epidermal cells were captured in 6-d-old seedlings. Upper left panel shows magnified cells with CaTLP1-YFP fluorescence, while right panel shows the brightfield image of cell patterns. Lower left panel shows fluorescence of PI-stained cell walls and nucleus, whereas right panel shows fluorescence of PI-stained cells overlaid onto brightfield images of cells expressing CaTLP1-YFP. (b) Localization of CaTLP1-YFP in Arabidopsis primary roots under dehydration. The seedlings were removed from MS plate and subjected to dehydration on glass slide for 15 mina Upper left panel shows yellow fluorescence of CaTLP1-YFP, while right panel shows brightfield image of roots. Lower left panel shows fluorescence of PI-stained cell walls and nucleus, whereas right panel shows fluorescence of PI-stained cells overlaid onto brightfield images of cells expressing CaTLP1-YFP. (c) Zoomed images of dehydrated cells expressing CaTLP1-YFP with overlaid images on brightfield (1^{st} and 2^{nd} sections) and PI-stained cells with overlaid images (3^{rd} and 4^{th} sections). The bars indicate the extent of resolutions.

FIG. 9 shows Molecular characterization of transgenic tobacco plants expressing CaTLP1. (a) PCR amplification of CaTLP1 using gene-specific primers. (b) Expression of CaTLP1 in transgenic plants as determined by RNA gel-blot analysis. Total RNA was extracted from wild-type and transgenic plants, and probed with [³²P]-labelled 0.5 kb 5′-CaTLP1 fragment. Ethidium bromide-stained rRNA is shown as loading control. (c) Western blot analysis of independent transgenic lines. Total protein was extracted from both wild-type and transgenic plants, and probed with anti-CaTLP1 polyclonal antibody. The representative Coomassie-stained gel shows uniform protein loading. (d) Southern blot analysis of CaTLP1-overexpressing plants. Genomic DNA of two independent transgenic events (T1 and T4) was digested with EcoRI that has no restriction site in the probe. The blot was hybridized with [³²P]-labelled GUS cDNA probe. Molecular weight marker in kb is indicated on the left.

FIG. 10 shows phenotypic screening of wild-type and CaTLP1-overexpressing tobacco plants. (a) Morphology of 15- to 45-d-old wild-type and transgenic tobacco plants; (b) root phenotype of 15-d-old plants; and (c) leaf-area. (d) Observation under scanning electron microscope (SEM) shows size of primary root cells (upper panels) and the leaf epidermal cells (lower panels). The bar indicates the extent of resolution. The representative T1 and T4 transgenic plants expressing CaTLP1 are shown. Days were counted after seedlings had been transplanted to soil. The error bars in the graphs represent the standard deviation of the values taken from three plants for each of two independent transgenic lines. *Significant difference (*P<0.05, **P<0.005) between control and given time point. Asterisks indicate significant difference with day 0 (P<0.05).

FIG. 11 shows comparative analysis of CaTLP1-overexpressing tobacco plants (T1 and T4) and the wild-type (WT) plants in terms of (a) biomass and (b) shoot length. Data represent means SD of three measurements. The bar numbers are representatives of the individual transgenic events.

FIG. 12 shows differential stress-response of wild-type and CaTLP1-overexpressing plants. (a) Observation of chlorosis due to osmotic or oxidative stress on the leaf-discs subjected to sorbitol, NaCl, H₂O₂, and menadione treatments. Leaf-discs (1 cm²) of 30-d-old wild-type and transgenic tobacco plants were floated on different concentrations of the solutions as indicated. The discs floated on sterile distilled water served as experimental control. (b) Quantitation of chlorophyll content in leaf-discs of wild-type and transgenic plants under progressive osmotic stress of 250 mM NaCl. The experiments were done in triplicates (n=3), and average mean values were plotted against duration of stress. (c) Phenotypic appearance of transgenic plants compared to wild-type plants treated with 1 μM ABA. (d) Fresh weight of 3-week-old seedlings grown in MS media supplemented with ABA compared to the fresh weight of the seedlings in unstressed condition expressed as percent relative fresh weight. The error bars in the graphs represent the standard deviation of the values taken from three plants for each of two independent transgenic lines. *Significant difference (*P<0.05, **P<0.005) between control and given time point. Asterisks indicate significant difference with day 0 (P<0.05).

FIG. 13 shows improved dehydration tolerance in transgenic tobacco plants expressing CaTLP1. (a) Dehydration-induced temporal changes of net photosynthesis in wild-type and transgenic plants. (b) Measurement of water loss in the excised leaves of wild-type and transgenic plants. The fresh weight (FW) was measured at different time intervals as indicated, and water loss was calculated from the decrease in FW compared with time zero. Mean values were normalised to the leaf-area of 10 leaves for each of three independent experiments. (c) Phenotypic appearance of wild-type and transgenic plants, wherein the rate of water-loss was compared. (d) Changes in transpiration rate of transgenic and wild-type plants in a time-dependent manner under dehydration. The dehydration treatment was administered as described in ‘Materials and methods’. Values are expressed as mean from five independent leaves of three randomly chosen plants. (e) Phenotypic appearance of wild-type and transgenic plants, wherein the rate of photosynthesis and transpiration were measured.

FIG. 14 shows the nucleotide and predicted amino acid sequences of CaTLP1. Coding and non-coding regions are shown in upper and lower case letters, respectively. The sequence starts with transcription site (+1). Single-letter codes for amino acids are given below the coding region. The introns are indicated by dotted underline. The positions of start (ATG) and stop (TAG) codons are indicated in bold letters. The F-box and Tub domains are underlines by solid and dotted lines, respectively.

### DETAILED DESCRIPTION OF THE INVENTION

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

### DEFINITIONS

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

As used in the specification and the claims, the singular forms “a”, “an”, and “the” include plural referents unless the context clearly dictates otherwise.

The terms “comprise” “comprising” “including” “containing” “characterized by” and grammatical equivalents thereof are used in the inclusive, open sense, meaning that additional elements may be included. It is not intended to be construed as “consists of only.”

The term “polypeptide” is defined as an amino acid sequence comprising a plurality of consecutive polymerized amino acid residues e.g., at least about 15 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

The term “Protein” refers to an amino acid sequence, oligopeptide, peptide, polypeptide or portions thereof whether naturally occurring or synthetic. “Proteins” as used herein, includes a wide variety of peptide-containing molecules, including monomeric, dimeric, multimeric, heterodimeric, heterotrimeric, and heterotetrameric proteins; disulfide bonded protein; glycosylated proteins; helical proteins; and α and β sheet-containing proteins.

The term “recombinant” as used herein, means that a molecule (e.g., a nucleic acid or a polypeptide) has been artificially or synthetically (i.e., non-naturally) altered by human intervention. The alteration can be performed on the molecule within, or removed from, its natural environment or state.

The term “recombinant protein” or “recombinant polypeptide” as used herein, refers to a protein molecule which is expressed using a recombinant DNA molecule.

The term “recombinant polynucleotide” is defined as a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

The term “expressing a protein” or “protein expression” means the function of a cell to transcribe recombinant DNA to mRNA and translate the mRNA to a protein. For expression the recombinant DNA usually includes regulatory elements including 5′ regulatory elements such as promoters, enhancers, and introns; other elements can include polyadenylation sites, transit peptide DNA, markers and other elements commonly used by those skilled in the art. Promoters can be modulated by proteins such as transcription factors and by intron or enhancer elements linked to the promoter. Promoters in recombinant polynucleotides can also be modulated by nearby promoters.

The term “host cell” refers to a microbial cell such as bacteria and yeast or other suitable cell including animal or a plant cell which has been transformed to express the homologous and heterologous proteins of interest. Host cells which are contemplated by the present invention include those in which the over-expressed proteins by the cell are sequestered in refractile bodies. An exemplary host cell is E. coli BL21 (DE3), which has been transformed to effect expression of the desired recombinant protein.

The term “wild type plant” refers to the form of the plant that occurs most frequently in nature. It is distinguished from mutant forms that may result from selective breeding.

A transgenic “plant cell” means a plant cell that is transformed with stably-integrated, non-natural, recombinant polynucleotides, e.g. by Agrobacterium-mediated transformation or by bombardment using micro particles coated with recombinant polynucleotides. A plant cell of this invention can be an originally-transformed plant cell that exists as a microorganism or as a progeny plant cell that is regenerated into differentiated tissue, e.g. into a transgenic plant with stably-integrated, non-natural recombinant polynucleotides in its chromosomal DNA, or seed or pollen derived from a progeny transgenic plant.

A “transgenic” plant or seed means one whose genome has been altered by the stable incorporation of recombinant polynucleotides in its chromosomal DNA, e.g. by transformation, by regeneration from a transformed plant from seed or propagule or by breeding with a transformed plant. Thus, transgenic plants include progeny plants of an original plant derived from a transformation process including progeny of breeding transgenic plants with wild type plants or other transgenic plants.

The term “alignment” refers to a number of nucleotide bases or amino acid residue sequences aligned by lengthwise comparison so that components in common (i.e., nucleotide bases or amino acid residues) may be visually and readily identified. The fraction or percentage of components in common is related to the homology or identity between the sequences.

The term “identity” or “similarity” refers to sequence similarity between two or more polynucleotide sequences, or two or more polypeptide sequences, with identity being a more strict comparison. “Sequence similarity” refers to the percentage of bases that are similar in the corresponding positions of two or more polynucleotide sequences. A degree of homology or similarity of polypeptide sequences is a function of the number of similar amino acid residues at positions shared by the polypeptide sequences. Two or more sequences can be anywhere from 0-1.00% similar, or any integer value there between. Identity or similarity can be determined by comparing a position in each sequence that may be aligned for purposes of comparison.

The present invention provides a polynucleotide encoding the protein having the amino acid sequence as set forth in SEQ ID NO: 5. The present invention also provides a recombinant DNA construct comprising the polynucleotide encoding the protein having the amino acid sequence as set forth in SEQ ID NO: 5 operably linked to a promoter, a recombinant vector comprising the recombinant DNA construct, a host cell comprising the polynucleotide of the present invention that encodes the protein having the amino acid sequence as set forth in SEQ ID NO: 5, a process for producing a transformed plant cell, plant or a part thereof over-expressing abiotic stress responsive protein having amino acid sequence as set forth in SEQ ID NO: 5, a transgenic plant, plant cell or progeny thereof over-expressing abiotic stress responsive protein having amino acid sequence as set forth in SEQ ID NO: 5, wherein the transgenic plant, plant cell or progeny thereof is more tolerant to abiotic stress than a wild type plant and a transgenic seed or progeny obtained from the transgenic plant, plant cell or progeny thereof, wherein said seed or the progeny over-expresses abiotic stress responsive protein having amino acid sequence as set forth in SEQ ID NO: 5 and is more tolerant to abiotic stress than a wild type plant.

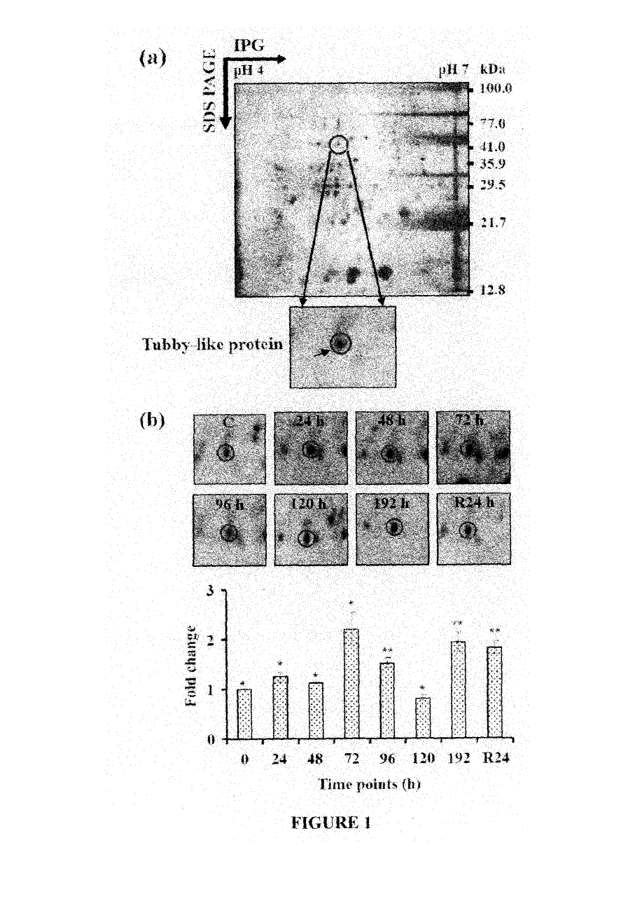

The invention disclosed in the present specification investigated the temporal changes in the proteome of C. arietinum that led to the identification of a tubby-like protein (TLP), designated CaTLP1, putatively involved in dehydration. A critical analysis of the differential proteome of extracellular matrix of chickpea under dehydration revealed a spot representing tubby-like protein (TLP) that was found to be highly regulated under dehydration (FIG. 1a). Cell wall proteins were electrofocused onto 13 cm IPG strip (pH 4-7) and resolved on 12.5% (w/v) SDS-PAGE. Gel images were analyzed using PDQuest, version 7.2.0, and the TLP spot in normalized densities among different time intervals was identified. The gel section containing the candidate spot is magnified in the lower panel of FIG. 1a. A threshold level of expression of TLP was observed in unstressed tissues, indicating its role in plant growth and development. Under dehydration, expression of the protein reached maximal level at 72 hours and decreased during 96 hours to 1.20 hours, probably due to feedback inhibition by the accumulated protein itself. The expression level was further elevated at later stages of dehydration and also at rehydrated stage (FIG. 1b).

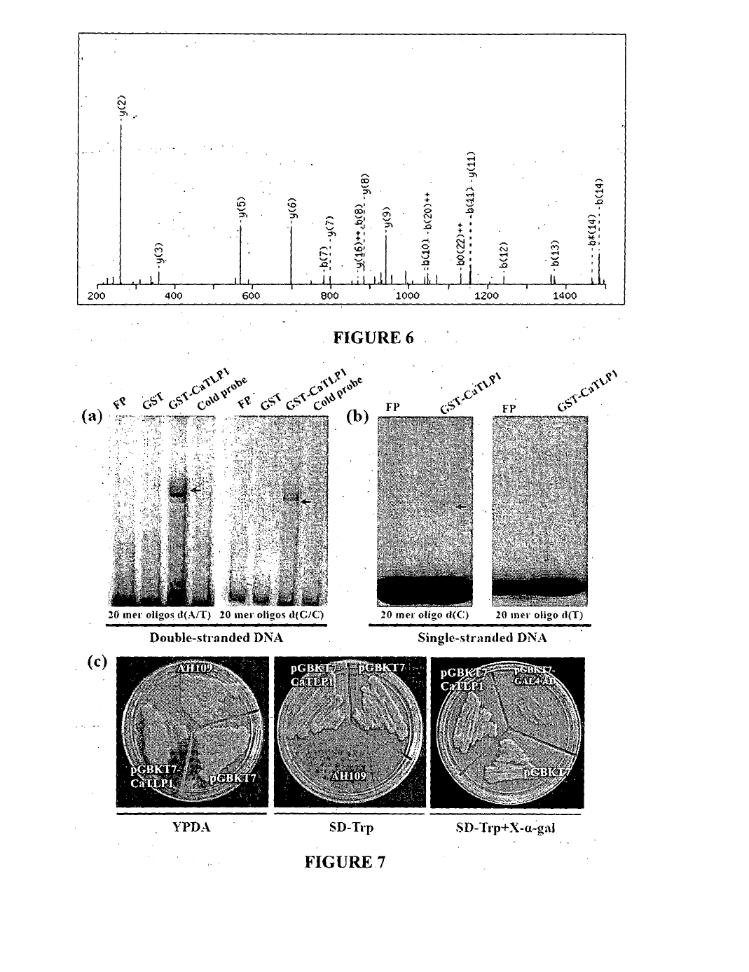

The 3′-rapid amplification of cDNA using degenerate primers having sequences as set forth in SEQ ID NO: 1 and SEQ ID NO: 2, designed from peptide sequence tags, yielded a 1.0 kb partial clone homologous to a chickpea EST sequence, gi-7635492. The coding region of the gene as set forth in SEQ ID NO: 4 was isolated, cloned in pGEM-T vector and named as pCaTLP1. Further, the full-length ORF of CaTLP1 was cloned in-frame with GST cDNA in pGEX4T-2 and expressed in Escherichia coli, BL21 (DE3) cells, which encode the fusion protein, GST-CaTLP1 with an estimated molecular mass of 72 kDa (FIG. 5). The expressed protein was confirmed by mass spectrometry (FIG. 6). In silico analysis revealed that the actual transcript size of the polynucleotide encoding CaTLP1 protein was 1,722 bp, as set forth in SEQ ID NO: 7, with a coding region of 1,236 bp as set forth in SEQ ID NO: 4, and 215-bp 5′- and 271-bp 3′-UTRs, respectively.

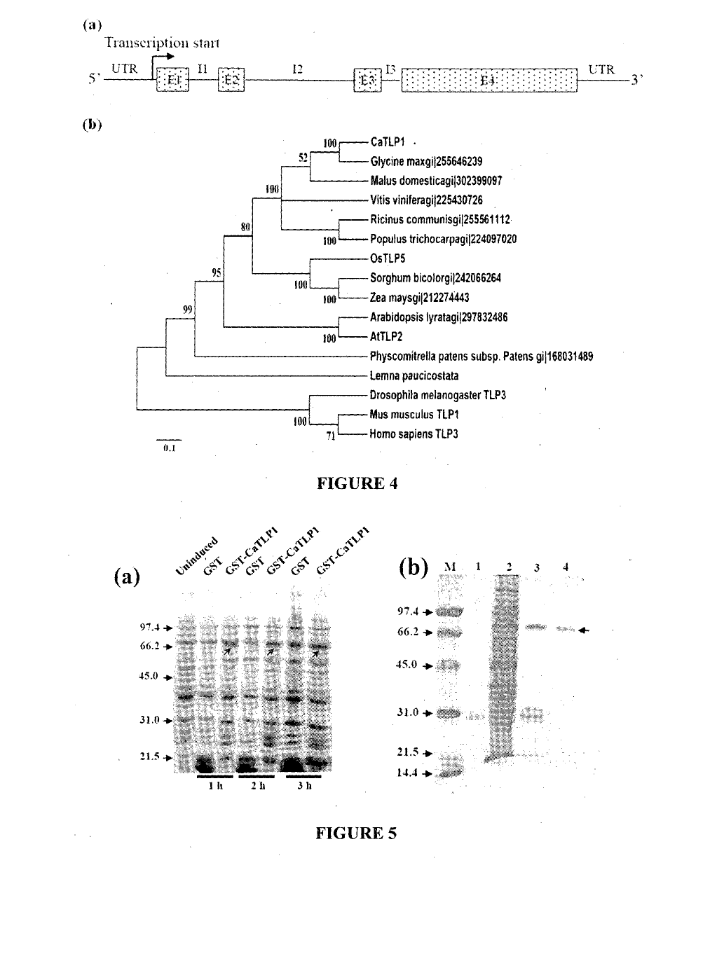

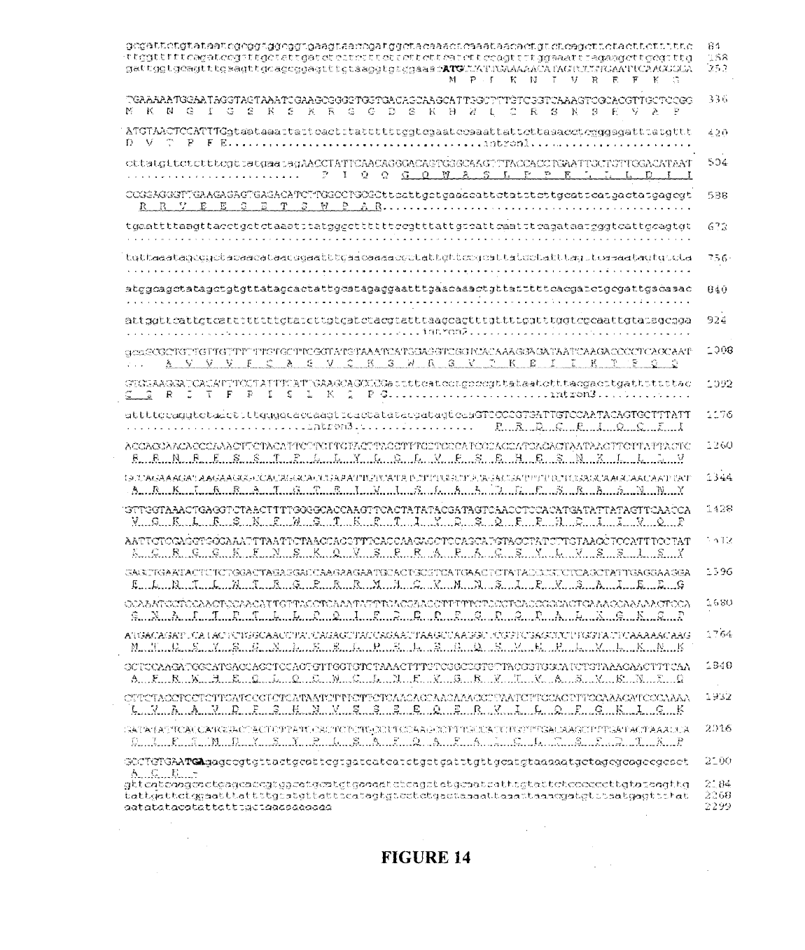

The CaTLP1 genomic DNA is 2,299 bp (SEQ ID NO: 6), indicating the presence of introns. The comparison of the full-length cDNA sequence with the corresponding genomic DNA sequence revealed that the coding region of the CaTLP1 is interrupted by three introns (FIG. 4). The deduced protein sequence of CaTLP1 having amino acid sequence as set forth in SEQ ID NO: 5 was analysed. The domain search (http://www.expasy.org/prosite) of the CaTLP1 protein revealed a well-conserved Tub-domain (PF01167) of 294 residues spanning 117-411 aa at the C-terminal region and F-box domain (Pfam00646) of 55 residues spanning 51-106 aa at the N-terminal region The CaTLP1 gene has revealed the presence of three intron regions at positions 352-446 (Intron 1), 543-927 (Intron 2) and 1048-1144 (Intron 3). The exons are prevalent in positions 216-351 (Exon 1), 447-542 (Exon 2), 928-1047 (Exon 3) and 1145-2028 (exon 4) positions of the CaTLP1 gene in C. arietinum (FIG. 14).

To characterize the CaTLP1 gene in more detail, Southern blotting was performed, which could be used to obtain preliminary information on the size and organization of the corresponding genomic sequence and the number of gene copies. Genomic DNA of chickpea was digested with different restriction enzymes and blotted to nylon membrane. The blot was hybridized with [³²P]-labelled 314-bp CaTLP1 fragment under high stringency conditions. The restriction enzymes SspI and SacI, having single site in, the probe region, yielded two bands, whereas HindIII, EcoRI, and BamHI having no restriction sites yielded a single band, indicating the presence of single copy of the CaTLP1 (FIG. 3). However, Southern analysis when performed with 3′-conserved region of the CaTLP1 cDNA as probe, displayed multiple bands. These results confirm that the genes encoding TLPs in the chickpea genome are organized in low copy multigene family.

The CaTLP1 binds avidly to double-stranded DNA, but poorly to single-stranded DNA. Gel shift assay revealed band shift with GST-CaTLP1 fusion protein in case of both the double-stranded DNA molecules, while no shift was observed with the purified GST (FIG. 7). The binding of radio-labelled probe was effectively competed out by addition of 100-fold excess of cold oligonucleotides. Gel shift assay using single-stranded d(C20) and d(T20) oligonucleotides as probe yielded no or little band shift (FIG. 7). This specificity indicates that the binding is not the result of non-specific electrostatic interactions, rather dependent on specific determinants, characteristic of double-stranded DNA.

It is known that there is a close relation between physiological function of a protein and its intracellular location. In mammalian cells, tubby protein is reported to be localized in the nucleus as well as in plasma membrane, and shown to have a direct link in G-protein signalling that regulate the gene expression. Mutation studies in the tubby-like protein family gene showed significant abnormalities and phenotypes that do not always overlap despite overlapping expression patterns. Therefore, the TLPs are multifunctional proteins, and may play multiple independent functional roles.

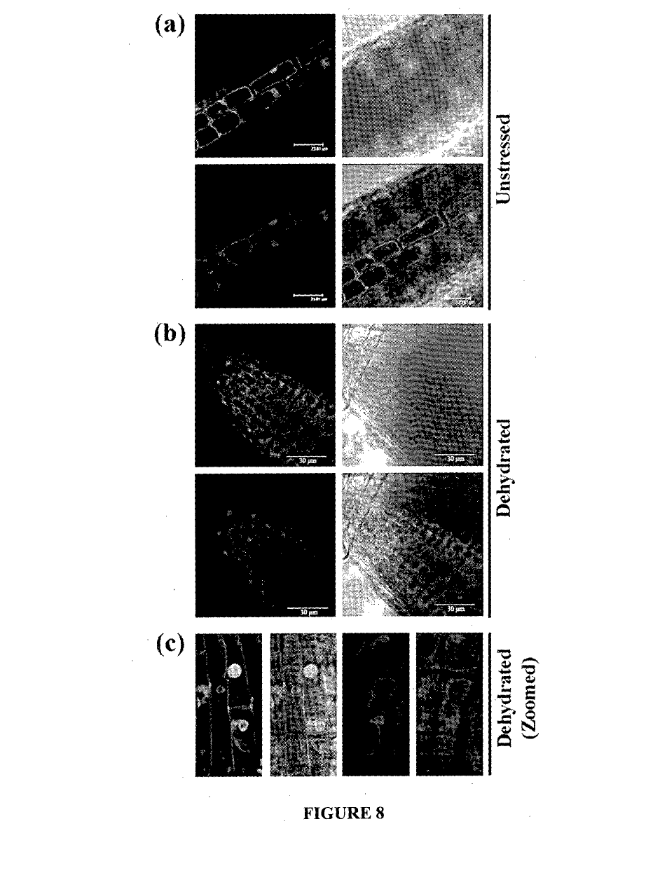

This is the first report demonstrating the localization of tubby protein in plants. CaTLP1 is localized predominantly in the cell wall and nucleus (FIG. 8). An examination of the amino acid sequence using Subcellular Localization (SCL) Predictor CELLO v.2.5 revealed that CaTLP1 is duly localized to the extracellular space as well as nucleus. The multi-organellar localization of CaTLP1 can be attributed to its role in early stress response primarily for perceiving the signals at the cell wall, and then driving them by nuclear translocation. The regulation of CaTLP1 function through control of dual localization is of interest because this characteristic feature is well suited for its effective functioning under stress conditions.

The characteristic properties of CaTLP1, its nuclear translocation and DNA-binding activity support, at least partly, a putative transcription factor. However, its inability to induce the transcription of reporter gene may account for the absence of transactivation domain in CaTLP1. Unlike highly diverse N-terminal sequence of animal TLPs, CaTLP1 contains conserved F-box domain. The role of F-box is established as cell surface receptor and transcription modulator. These proteins use broad and different mechanisms for target recognition, the most common mechanism being the formation of a SkpI/Cullin/F-box (SCF) complex. The presence of the conserved F-box domain in CaTLP1 suggests a key role in stress tolerance, possibly by protein-protein interaction. CaTLP1 presumably serves as a molecular sensor. Following the possible signal transduction initiating events, it translocates to the nucleus to carry out targeted biological function. Probably CaTLP1 binds to DNA and interacts with the regulator with the help of Tub and F-box domains, respectively. F-box domain may either regulate the activity or recruit other regulators at the promoter that can effectively switch on and off the downstream gene expression.

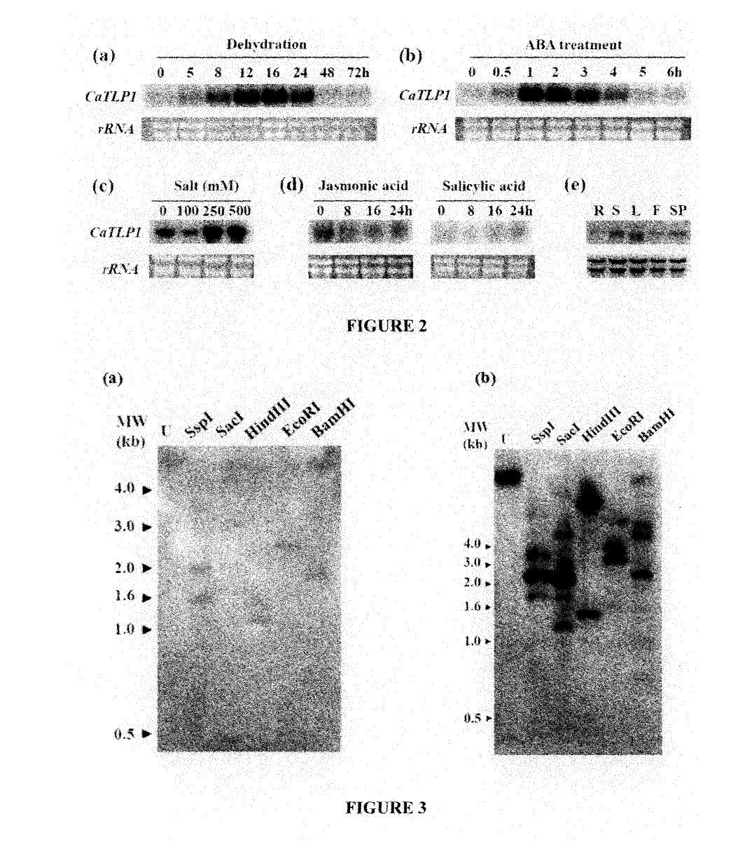

The organ specificity of CaTLP1 expression revealed low but detectable expression in roots, flowers and pods but substantially higher expression in vegetative organs such as stems and leaves, suggesting a possible synergistic quantitative relationship of CaTLP1 to growth and development of the plant (FIG. 2).

The dehydration response of CaTLP1, evaluated using the Northern analysis revealed that the CaTLP1 transcripts were induced within 5 hours of dehydration with maximum accumulation at 12 hours that persisted up to 16 hours. However, the transcripts showed a drastic decrease after 24 hours and reached the background level at 48 hours (FIG. 2). The CaTLP1 was induced by abscisic acid (ABA) after 30 minutes of treatment and reached saturation levels at 1 hour that persisted up to 3 hours, but dropped to background level after 4 hours of treatment (FIG. 2). Salt stress experiments with NaCl revealed that CaTLP1 transcripts were most strongly induced at 250 mM NaCl (FIG. 2) but not at lower concentrations of NaCl. CaTLP1 possibly has little or no role in pathostress signalling as indicated by studying the pathostress response in plants induced by jasmonic acid (JA) and salicylic acid (SA), compounds which are essential in the pathogen- and wound-signalling pathways. Chickpea seedlings treated with JA and SA showed very little change in the accumulation of CaTLP1 transcripts compared with non-treated seedlings (FIG. 2).

Transient expression analysis of CaTLP1-YFP fusion protein in transfected tobacco cells showed fluorescence in both extracellular matrix and nucleus (FIG. 4b), as did stable expression in transgenic Arabidopsis plants (FIGS. 5a to 5c). Many stress-responsive proteins are known to shuttle between different sub-cellular compartments, albeit their role in signal transduction mechanisms remains largely unexplored. The transgenic plants subjected to osmotic stress revealed relatively strong YFP signals in the nucleus compared to the extracellular space (FIGS. 5b and 5c). The stress-induced preferential accumulation of CaTLP1 in the nucleus shows that the localization of the protein varies considerably under normal and stress conditions indicating a key role in stress response.

To investigate the potential of CaTLP1 to activate a large number of stress-responsive genes, the ORF of CaTLP1 was introduced in-frame with the GAL4 DNA-binding domain in yeast expression vector pGBKT7. The pGBKT7-CaTLP1 construct was then transformed into yeast strain AH109 harbouring MEL1 reporter gene. The colonies were observed for time-dependent colour development; but strangely, they remained white even after three days of incubation whereas the vector colonies turned blue (FIG. 7). This inability, to induce the transcription of reporter gene, may account for the absence of transactivation domain in CaTLP1. Nevertheless, the characteristic properties of CaTLP1, particularly stress-induced preferential accumulation in the nucleus and DNA-binding activity support it, at least partly, to be a putative transcription factor (FIGS. 7 and 8).

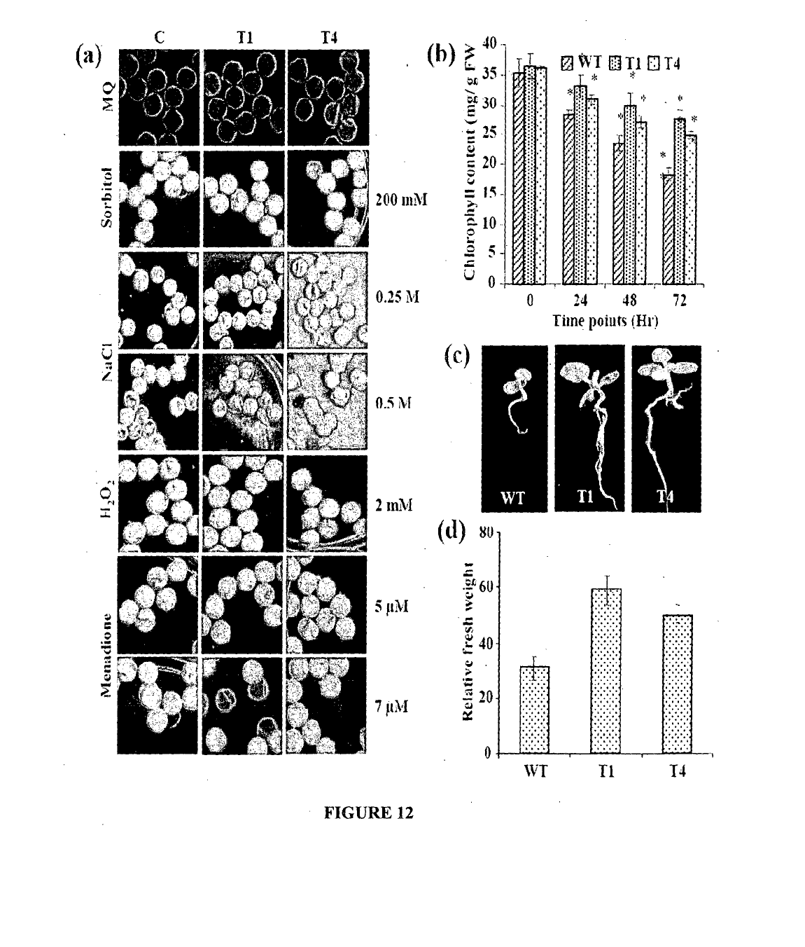

Transgenic tobacco plants constitutively expressing CaTLP1 under the control of CaMV35S promoter were evaluated for their response to stress. Over-expression of CaTLP1 in transgenic tobacco plants rendered improved tolerance to dehydration, salinity and oxidative stresses. There were significant increases in root and leaf development, net photosynthesis, and plant biomass in CaTLP1-overexpressing transgenic lines (FIGS. 7a, 7b and 7c). Molecular genetic analysis and recapitulation experiments showed that the enhanced stress tolerance is caused by the preferential expression of the protein in the nucleus suggesting important roles of CaTLP1 in stress-responsive pathways and in the regulation of plant development. Transgenic plants showed very little chlorosis compared to the wild-type plants (FIG. 12). Transgenic plants also retained higher percentage of chlorophyll compared with that of wild-type plants (FIG. 12). The higher retention rate of chlorophyll in the transgenic plants even after 72 hours of incubation confirms the observed phenotypic differences (FIG. 12). Stress-induced loss of chlorophyll was lower in CaTLP1-overexpressing plants, reflecting their better ability to withstand such stress. The relative fresh weight of transgenic plants was higher than the wild type plants under abscisic acid (ABA)-mediated stress (58% in T1, 50% in T4, and 31% in the wild-type plants) as shown in FIG. 12. CaTLP1 along with ABA plays a significant role in dehydration and salt-stress responsive pathways in plants.

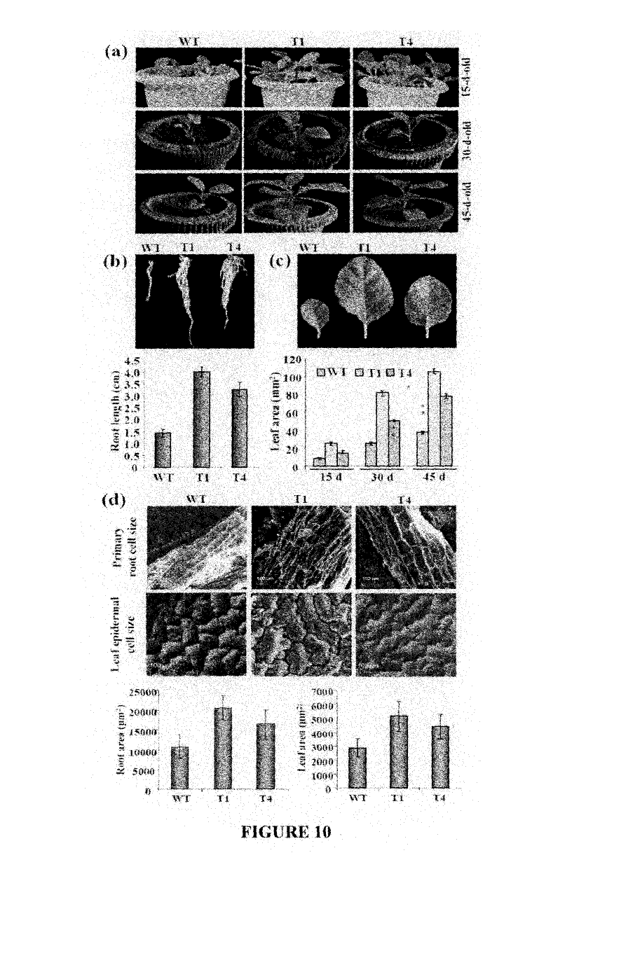

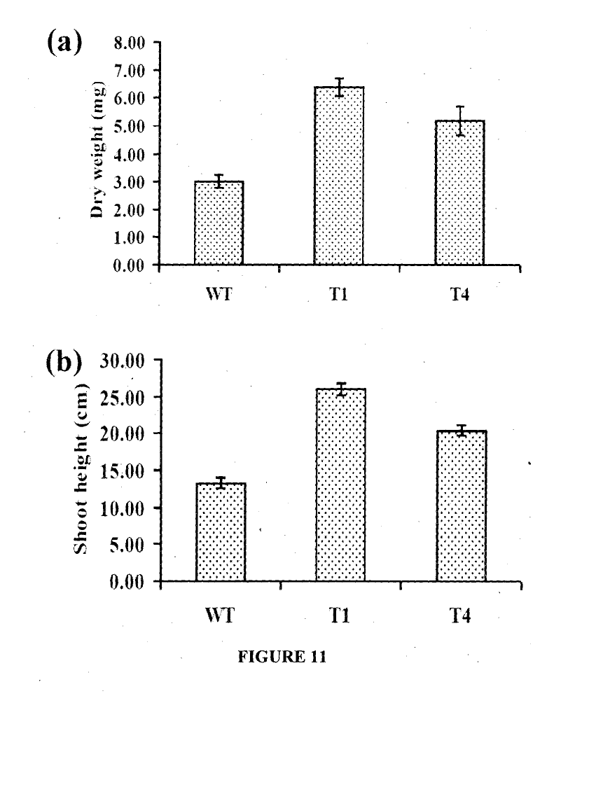

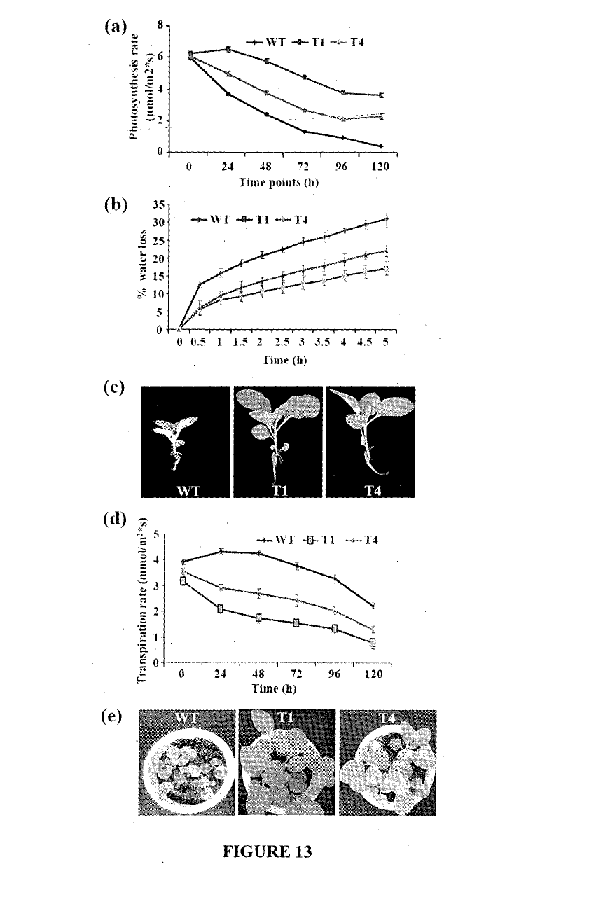

The expression of CaTLP1 is associated with dehydration tolerance by maintaining turgidity and relative water content. Under progressive dehydration, the photosynthetic rate in transgenic plants remained unaltered in initial 24 hours, decreased linearly up to 96 hours, and sustained thereafter. In contrast, the decrease in photosynthetic rate in wild-type plants was found to be proportional to the duration of stress treatment. In wild-type plants, photosynthetic rate reduced to 12% after 120 hours of dehydration, while the rates were 60% and 50% in transgenic T1 and T4, respectively (FIG. 13). During the time-course of dehydration, the detached leaves of transgenic plants lost water more slowly as compared to wild-type plants (FIGS. 10b and 10c). Without wishing to bind to a specific theory, the inventors believe that the resistance to dehydration in transgenic plants could be attributed, at least in part, to the ability of the transgenic plants to respond to stress-induced stomatal changes by efficiently closing their stomata and reducing transpiration rates. It is possible that CaTLP1 might influence stomatal closure in a similar fashion in order to minimize water loss, which is corroborated by higher expression of CaTLP1 in. leaves (FIG. 2). CaTLP1 may be associated with synaptic function in stress tolerance in plants (FIG. 13). This possibility is exemplified by the observation of reduced transpiration rate in transgenic plants compared to wild-type plants subjected to dehydration (FIG. 13).

The present invention discloses the role of a tubby-like protein, CaTLP1, in plant growth and development and in stress tolerance. The sequence of CaTLP1 displayed high similarity with the tubby genes earlier reported in Arabidopsis and rice. Over-expression of CaTLP1 in transgenic tobacco plants showed enhanced growth and development compared to wild-type plants. The CaTLP1 binds to double-stranded DNA but is incapable of transcriptional activation. The gene structure and organization of CaTLP1 revealed that CaTLP1 is involved in osmotic stress-responsive pathway in plants. The transcripts are strongly expressed in vegetative tissues but weakly in reproductive tissues. CaTLP1-overexpressing plants showed improved tolerance to dehydration, high salinity, and oxidative stress indicating its possible role in multivariate stress-responsive pathways. CaTLP1 is up regulated by dehydration and high salinity, and also by abscisic acid (ABA) treatments suggesting that its stress-responsive function might be associated with ABA-dependent network. The present invention provides new insights into the underlying mechanism of action of plant TLPs and also facilitates the targeted genetic manipulation in crop plants to improve stress tolerance. The polynucleotide and polypeptide sequence disclosed in the present specification also facilitates growth and development in the plant.

The present invention is not to be limited in scope by the specific embodiments described herein, which are intended for the purposes of exemplification only. Functionally-equivalent products, compositions, and methods are clearly within the scope of the invention, as described herein.

In an embodiment of the present invention, there is provided a polynucleotide encoding the protein having the amino acid sequence as set forth in SEQ ID NO: 5.

An embodiment of the present invention provides a polynucleotide encoding the protein having the amino acid sequence as set forth in SEQ ID NO: 5, wherein the nucleotide sequence of the polynucleotide has at least 90% sequence identity with the nucleotide sequence as set forth in SEQ ID NO: 4, SEQ ID NO: 6, or SEQ ID NO: 7.

Another embodiment of the present invention provides a polynucleotide encoding the protein having the amino acid sequence as set forth in SEQ ID NO: 5, wherein the nucleotide sequence of the polynucleotide is as set in SEQ ID NO: 4, SEQ ID NO: 6, or SEQ ID NO: 7.

In yet another embodiment of the present invention, there is provided a polynucleotide encoding the protein having the amino acid sequence as set forth in SEQ ID NO: 5, wherein the polynucleotide is responsible for conferring enhanced tolerance to abiotic stress in a transformed plant as compared to an untransformed plant.

In still another embodiment of the present invention, there is provided a polynucleotide encoding the protein having the amino acid sequence as set forth in SEQ ID NO: 5, wherein the polynucleotide is responsible for conferring enhanced tolerance to abiotic stress in a transformed plant as compared to an untransformed plant, wherein the abiotic stress is selected from the group consisting of dehydration stress, salinity stress, oxidative stress, osmotic stress, drought stress, and phytohormone stress.

In an embodiment of the present invention, there is provided a polynucleotide encoding the protein having the amino acid sequence as set forth in SEQ ID NO: 5, wherein the polynucleotide is responsible for enhanced plant growth and development in a transformed plant as compared to an untransformed plant.

Another embodiment of the present invention provides a recombinant DNA construct comprising the polynucleotide as claimed in claim 1; or the polynucleotide having the nucleotide sequence encoding the protein having the amino acid sequence as set forth in SEQ ID NO: 5; or the polynucleotide having the nucleotide sequence asset forth in SEQ ID NO: 6; or the polynucleotide having the nucleotide sequence as set forth in SEQ ID NO: 7, wherein the polynucleotide is operably linked to a promoter.

An embodiment of the present invention provides a recombinant vector comprising a recombinant DNA construct comprising the polynucleotide encoding the protein having the amino acid sequence as set forth in SEQ ID NO: 5; or the polynucleotide having the nucleotide sequence as set forth in SEQ ID NO: 4; or the polynucleotide having the nucleotide sequence as set forth in SEQ ID NO: 6; or the polynucleotide having the nucleotide sequence as set forth in SEQ ID NO: 7, wherein the polynucleotide is operably linked to a promoter.

In yet another embodiment of the present invention, there is provided a recombinant host cell comprising a polynucleotide encoding the protein having the amino acid sequence as set forth in SEQ ID NO: 5 or the recombinant vector comprising a recombinant DNA construct comprising the polynucleotide encoding the protein having the amino acid sequence as set forth in SEQ ID NO: 5; or the polynucleotide having the nucleotide sequence, as set forth in SEQ ID NO: 4; or the polynucleotide having the nucleotide sequence as set forth in SEQ ID NO: 6; or the polynucleotide having the nucleotide sequence as set forth in SEQ ID NO: 7, wherein the polynucleotide is operably linked to a promoter.

In still another embodiment of the present invention, there is provided a recombinant host cell comprising a polynucleotide encoding the protein having the amino acid sequence as set forth in SEQ ID NO: 5 or the recombinant vector comprising a recombinant DNA construct comprising the polynucleotide encoding the protein having the amino acid sequence as set forth in SEQ ID NO: 5; or the polynucleotide having the nucleotide sequence as set forth in SEQ ID NO: 4; or the polynucleotide having the nucleotide sequence as set forth in SEQ ID NO: 6; or the polynucleotide having the nucleotide sequence as set forth in SEQ ID NO: 7, wherein the polynucleotide is operably linked to a promoter, wherein the host cell is selected from the group consisting of E. coli, Agrobacterium, yeast and, plant cell.

In an embodiment of the present invention, there is provided a polypeptide having amino acid sequence as set forth in SEQ ID NO: 5, wherein expression of the polypeptide is responsible for conferring enhanced tolerance to abiotic stress in a transformed plant as compared to an untransformed plant.

An embodiment of the present invention provides a method for producing a transformed plant cell, plant, or a part thereof over-expressing a protein having the amino acid sequence as set forth in SEQ ID NO: 5, wherein the method comprises: transforming the plant cell, plant, or part thereof with a polynucleotide encoding the protein having the amino acid sequence as set forth in SEQ ID NO: 5, and selecting the transformed plant cell, plant or part thereof, wherein the transformed plant cell, plant, or part thereof is more tolerant to abiotic stress than an untransformed plant.

Another embodiment of the present invention provides a method for producing a transformed plant cell, plant, or a part thereof over-expressing a protein having the amino acid sequence as set forth in SEQ ID NO: 5, wherein the method comprises: transforming the plant cell, plant, or part thereof with a polynucleotide encoding the protein having the amino acid sequence as set forth in SEQ ID NO: 5, and selecting the transformed plant cell, plant or part thereof, wherein the transformed plant cell, plant, or part thereof is more tolerant to abiotic stress than an untransformed plant, wherein the polynucleotide is as set forth in SEQ ID NO: 4.

In still another embodiment of the present invention, there is provided a method for producing a transformed plant cell, plant, or a part thereof over-expressing a protein having the amino acid sequence as set forth in SEQ ID NO: 5, wherein the method comprises: transforming the plant cell, plant, or part thereof with a polynucleotide encoding the protein having the amino acid sequence as set forth in SEQ ID NO: 5, and selecting the transformed plant cell, plant or part thereof, wherein the transformed plant cell, plant, or part thereof is more tolerant to abiotic stress than an untransformed plant, wherein the abiotic stress is selected from the group consisting of dehydration stress, salinity stress, oxidative stress, osmotic stress, drought stress, and phytohormone stress.

An embodiment of the present invention provides a method for producing a transformed plant cell, plant, or a part thereof over-expressing a protein having the amino acid sequence as set forth in SEQ ID NO: 5, wherein the method comprises: transforming the plant cell, plant, or part thereof with a polynucleotide encoding the protein having the amino acid sequence as set forth in SEQ ID NO: 5, and selecting the transformed plant cell, plant or part thereof, wherein the transformed plant cell, plant, or part thereof is more tolerant to abiotic stress than an untransformed plant, wherein the plant is a monocotyledonous or dicotyledonous plant.

In an embodiment of the present invention, there is provided a method for producing a transformed plant cell, plant, or a part thereof over-expressing a protein having the amino acid sequence as set forth in SEQ ID NO: 5, wherein the method comprises: transforming the plant cell, plant, or part thereof with a polynucleotide encoding the protein having the amino acid sequence as set forth in SEQ ID NO: 5, and selecting the transformed plant cell, plant or part thereof, wherein the transformed plant cell, plant, or part thereof is more tolerant to abiotic stress than an untransformed plant, wherein the plant is selected from the group consisting of tobacco, potato, sweet potato, cassava, sugar-beet, Arabidopsis, brassica species, tomato, brinjal, capsicum, chilli, okra, cucurbit; melon, mulberry, banana, mango, papaya, alfalfa, grass, canola, sunflower, cotton, legume plant, groundnut, peanut, pea, soybean, chickpea, pigeon pea, mungbean, medicago, lotus, Petunia, rice, maize, wheat, rye, barley, oats, pearl millet, corn, sorghum, nuts, avocado, turmeric, saffron, ginger, garlic, onion, nutmeg, forage plant, fruit tree, and ornamental plant.

Another embodiment of the present invention provides a method for producing a transformed plant cell, plant, or a part thereof over-expressing a protein having the amino acid sequence as set forth in SEQ ID NO: 5, wherein the method comprises: transforming the plant cell, plant, or part thereof with a polynucleotide encoding the protein having the amino acid sequence as set forth in SEQ ID NO: 5, and selecting the transformed plant cell, plant or part thereof, wherein the transformed plant cell, plant, or part thereof is more tolerant to abiotic stress than an untransformed plant, wherein plant is transformed using a method selected from the group consisting of floral dip method, Agrobacterium mediated transformation method, protoplast mediated transformation, particle gun bombardment method, in planta transformation, and liposome mediated transformation method.

A transgenic plant, plant cell or progeny thereof over-expressing a protein having the amino acid sequence as set forth in SEQ ID NO: 5, wherein the transgenic plant, plant cell or progeny thereof is more tolerant to abiotic stress than an untransformed plant.

The transgenic plant, plant cell or progeny thereof over-expressing a protein having the amino acid sequence as set forth in SEQ ID NO: 5, wherein the transgenic plant comprises the polynucleotide sequence as set forth in SEQ ID NO: 4 or a recombinant DNA construct comprising the polynucleotide as claimed in claim 1; or the polynucleotide having the nucleotide sequence encoding the protein having the amino acid sequence as set forth in SEQ ID NO: 5; or the polynucleotide having the nucleotide sequence as set forth in SEQ ID NO: 6; or the polynucleotide having the nucleotide sequence as set forth in SEQ ID NO: 7, wherein the polynucleotide is operably linked to a promoter.

The transgenic plant, plant cell or progeny thereof over-expressing a protein having the amino acid sequence as set forth in SEQ ID NO: 5, wherein the transgenic plant is selected from a group consisting of tobacco, potato, sweet potato, cassava, sugar-beet, Arabidopsis, Brassica species, tomato, brinjal, capsicum, chilli, okra, cucurbit, melon, mulberry, banana, mango, vitis, papaya, alfalfa, grass, canola, sunflower, cotton, legume plant, groundnut, peanut, pea, soybean, chickpea, pigeon pea, mungbean, medicago, lotus, petunia, rice, maize, wheat, rye, barley, oats, pearl millet, corn, sorghum, nuts, avocado, turmeric, saffron, ginger, garlic, onion, nutmeg, forage plant, fruit tree and ornamental plant. Although the subject matter has been described in considerable detail with reference to certain preferred embodiments thereof, other embodiments are possible. As such, the spirit and scope of the appended claims should not be limited to the description of the preferred embodiment contained therein.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1

### Nucleic Acid Analysis and Construction of Phylogenetic Tree

### Plant Growth Conditions

Cicer arietinum L., (chickpea) seedlings were grown in pots containing a 2:1 w/w mixture of soil and soilrite in an environment-controlled growth room and maintained at 25±2° C. with 50±5% relative humidity under 16 h photoperiod (270 μmol m⁻² s⁻¹ light intensity).

### Nucleic Acid Analysis and Construction of Phylogenetic Tree

Genomic DNA of C. arietinum L., (chickpea) seedlings was extracted using DNeasy kit (Qiagen, USA). Aliquots of 10 μg DNA were digested with different restriction enzymes (SspI, SacI, HindIII, EcoRI and BamHI) and Southern blot was hybridized with [³²P]-labelled 314-bp fragment of CaTLP1.

In a separate set of experiment, RNA was isolated using Tripure reagent (Roche Applied Science), as recommended by manufacturer. The electrophoresis of nucleic acids was performed as described by Sambrook et al., 2001 (Molecular Cloning: A Laboratory Manual, Cold Spring Harbor Laboratory Press, NY), blotted overnight onto Hybond-N membrane (Amersham Biosciences, UK), and fixed at 1200 j/cm for 30 s using Stratalinker UV crosslinker (Stratagene). Immobilized nucleic acids were hybridized with [³²P]-labelled 314-bp CaTLP1 fragment amplified with internal primers having nucleotide sequence as set forth in SEQ ID NO: 1 and SEQ ID NO: 2. The cDNA sequence of CaTLP1 was determined by ABI Prism 3700 DNA Analyzer (Applied Biosystems, USA). The cDNA nucleotide sequence thus obtained is as set forth in SEQ ID NO: 4.

A phylogenetic tree was constructed from amino acid alignment by neighbour-joining method using the default parameters. Multiple alignments of proteins were performed with ClustalW program, and a phylogram was generated using the neighbor-joining algorithm of MEGA software, version 5.05 as shown in FIG. 2b. The numerical represents the bootstrap value. Scale bar indicates an evolutionary distance of 0.1 aa substitution per position in the sequence (E, exon; I, intron; and UTR, untranslated regions). Two major groups were apparent from the phylogram. Plant TLP family proteins clustered in a single group, suggesting that they might originate from a single ancestral gene. However, the organization of TLPs in different members within the plant group suggests an evolutionary divergence in this protein family (Yang et al. 2008). Interestingly, TLP1 of mouse, TLP3 of human and fruit flies clustered together forming a separate group. Furthermore, these members were found to be close neighbors of CaTLP1, showing the cross-kingdom similarity.

### Example 2

### Cloning and Expression of CaTLP1 Protein in E. coli Construction of Recombinant Vector

The full-length CaTLP1 gene was amplified from the genomic DNA isolated from C. arietinum using forward and reverse primers of size 25 bp as set forth in SEQ ID NO:10 and SEQ ID NO:11, respectively.

The PCR product of size 1,236 bp was purified using QIAquick gel extraction kit (QIAGEN, Germany). The purified PCR product was ligated into pGEX4T-2 expression plasmid (Amersham Biosciences, UK) in-frame with GST fusion protein utilizing BamHI and NotI restriction sites engineered into 5′- and 3′-ends, respectively, using T4 DNA ligase enzyme (New England Biolabs, UK) in 1:3 molar ratio of vector to insert, to yield the recombinant vector construct pGEXCaTLP1. The successfully ligated constructs were verified by nucleotide sequencing and was used for the transformation in Escherichia coli BL21(DE3) strain.

E. coli Transformation and Expression of the Recombinant Protein

The recombinant vector pGEXCaTLP1 was introduced into the competent E. coli BL21(DE3) cells by transforming chemically competent cell (Invitrogen) and its selection on LB Agar media (Invitrogen, USA) supplemented with 50 mg ml⁻¹ ampicillin antibiotic.

The GST-tagged protein was produced by inducing transformed cells with 0.5 mM isopropyl β-D-1-thiogalactopyranoside. The recombinant protein was purified from the bacterial lysates with chromatography using glutathione-sepharose beads (Amersham Biosciences, UK) as per the manufacturer's instructions.

### Mass Spectrometric Identification of the Recombinant Protein

The purified recombinant protein was digested using trypsin. Trypsin-digested peptides were analyzed by electrospray ionization time-of-flight mass spectrometry (LC/MS/TOF) using a Tempo Nanoflow MDLC system coupled to Q-STAR Elite spectrometer (Applied Biosystems, USA). The spectra was analysed to identify the protein of interest using Mascot sequence matching software (Matrix Science) (FIG. 6).

### Electrophoretic Mobility Shift Assay and Transactivation Analysis

Gel-shift assays were carried out using double-stranded 20 mer d(A/T) and d(G/C) and single-stranded 20 mer d(C₂₀) and d(T₂₀) [³²P]-labelled probes. 0.5 mg of recombinant protein was incubated in a reaction mix containing 25 mM MOPS (pH 8.0), 2 mM DTT, 120 mM KOAc, and 2 mM EDTA with 0.5 pmol of either [³²P]-labelled double- or single-stranded oligonucleotides for 30 min at 30° C. The complexes were resolved on 6% polyacrylamide gel and visualized by autoradiography (FIG. 7). The shift was competed out by 100-fold excess of cold oligos. FIG. 7 demonstrates preference of CaTLP1 for binding double-stranded DNA and single stranded DNA probes. The arrows in the figure represent the band-shifts.

To determine the activation domain, the coding sequence of CaTLP1 (SEQ ID NO: 4) was cloned in yeast expression plasmid pGBKT7 (Clontech Laboratories, USA) at NdeI-BamHI restriction site to express CaTLP1 fused to GAL4 DNA-BD. The construct was transformed into yeast strain AH109 (Cagney, Uetz and Fields 2000) that harbour MEL1 reporter gene. The GAL4 AD domain from the plasmid pGADT7 (Clontech Laboratories, USA) was cloned in-frame with the plasmid pGBKT7 and used as positive control, while pGBKT7 plasmid transformed in AH109 cells served as negative control. Yeast colonies harbouring no vector, empty vector (pGBKT7), and recombinant pGBKT7-CaTLP1 were grown on synthetically defined medium YPDA synthetic medium deficient in tryptophan and on SD media lacking tryptophan but supplemented with X-α-gal. The transformants were then analyzed for α-galactosidase expression. FIG. 7 shows the transactivation analysis of CaTLP1.

### Example 3

### Transient Expression Assay for Sub-Cellular Localization of CaTLP1 Tobacco Seedlings

CaTLP1-Enhanced Yellow fluorescent Protein (YFP) construct in pGWB441 gateway vector were used to transform Agrobacterium. The Agrobacterium strain was grown overnight in 10 ml liquid culture at 28° C. with vigorous agitation and the resulting bacteria were suspended into a buffer solution known as infiltration medium of 10 mM MES, 10 mM MgCl₂ and 150 μM acetosyringone, up to an OD₆₀₀=2.0 Agrobacterium-mediated transient expression assay was carried out in epidermal cells of tobacco leaves, and the localization of chimeric CaTLP1 protein fused to YFP was analyzed. The fluorescent signals of YFP were confined to the extracellular space and nucleus of transfected cells (FIG. 3). To verify that the fluorescent signals observed were indeed cell wall specific, the transfected cells were stained with propidium iodide which showed that the red fluorescence of propidium iodide co-localized with the YFP signals, confirming the localization of CaTLP1 to the extracellular space of transfected cells (lower left panel of FIG. 3).

### Plant Transformation

Arabidopsis seedlings were transformed with the CaTLP1-YFP construct in pGWB441 gateway vector by floral-dip method (Clough S J, and Bent A F (1998) Floral dip: a simplified method for Agrobacterium-mediated transformation of Arabidopsis thaliana. Plant J. 16: 735-43). The bacteria were grown to stationary phase in liquid culture at 25-28° C., 250 r.p.m. in sterilized YEP (10 g yeast extract, 10 g peptone, and 5 g NaCl per liter) supplemented with spectinomycin (100 mg ml⁻¹). Cultures were initiated with 1:100 dilution of primary overnight cultures and grown for roughly 18-20 h. Cells were harvested by centrifugation (5500 g) for 20 min at room temperature. The harvested cells were resuspended in inoculation medium [½ strength MS Medium (M-5519; Sigma Chemicals), 5.0% sucrose, 44 nM benzylamino purine (10 ml 1⁻¹ of a 1 mg ml⁻¹ stock in DMSO) and 0.008% Silwet L-77] pH adjusted to 5.7 to a final OD₆₀₀ of approximately 2.0. Arabidopsis plants were transformed by floral-dip method (Clough and Bent 1998). The primary roots of transformants were examined under confocal microscope (Leica Microsystems). A cell wall specific dye, propidium iodide (PI), was used to stain the cell wall of the primary root cells (FIG. 8).

### Example 4

### Production of CaTLP1-Overexpressing Transgenic Plants

Full-length coding sequence of CaTLP1 (SEQ ID NO: 4) was cloned into the polylinker site of the binary vector pBI121 (Clontech Laboratories, USA) under the control of CaMV35S promoter. The construct was mobilized into Agrobacterium tumefaciens strain EHA105. Agrobacterium-mediated transformation of tobacco (Nicotiana tabacum L. var. Petit Havana) was carried out using standard protocol prevalent in the art (Hobbs S L, Kpodar P, DeLong C M (1990) The effect of T-DNA copy number, position and methylation on reporter gene expression in′tobacco transformants. Plant Mol Biol 15: 851-64).

Regenerated shoots were rooted on phytohormone-free rooting medium supplemented with cefotaxime (250 mg/L) and kanamycin (100 mg/L), transferred to soil, and grown in standardized greenhouse conditions. Seventeen independent transgenic events harboring CaTLP1 (SEQ ID NO: 4) were grown for further analyses. The wild-type and the transgenic tobacco plants were studied parallely in the same growth room and comparative morpho-anatomical, molecular, and physiological analyses were carried out. Morpho-anatomical studies were accomplished by scanning electron microscopy (EVO LS10, Carl Zeiss) (FIG. 10).

### Example 5

### Over-Expression of CaTLP1 Confers Stress Tolerance in Transgenic Plants

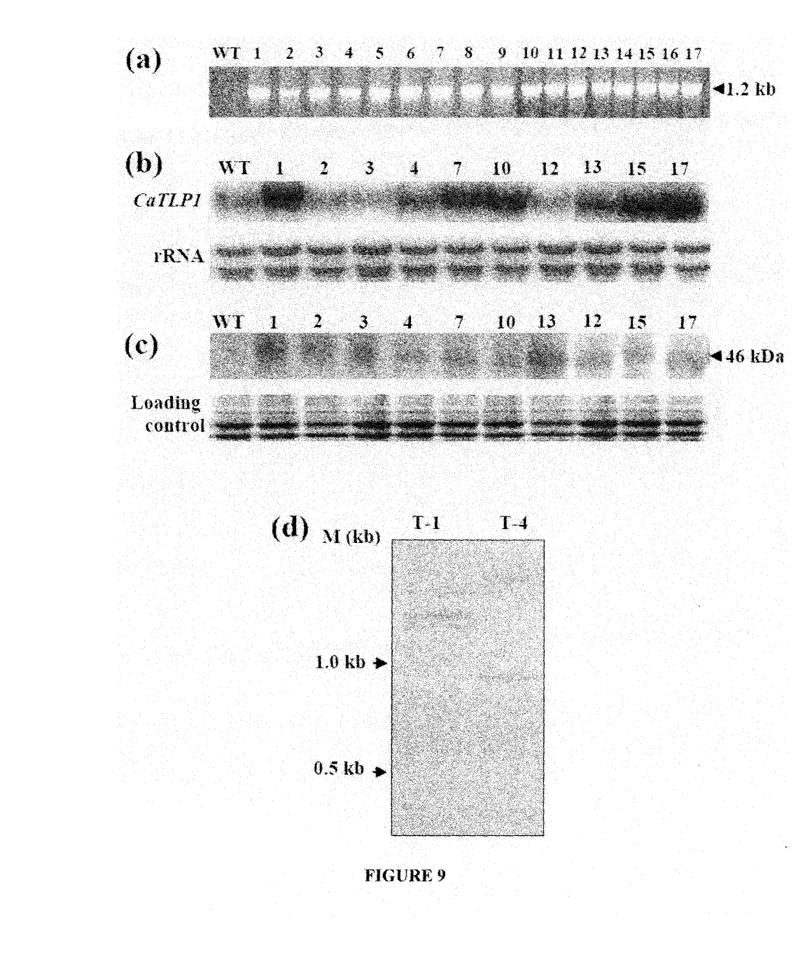

Transgenic tobacco plants constitutively expressing CaTLP1 (SEQ ID NO: 5) under the control of CaMV35S promoter was evaluated for its response to stress. A total of 17 independent transgenic events were confirmed by PCR analysis with primers having sequences as set forth in SEQ ID NO: 10 and SEQ ID NO: 11, the transcripts by Northern blot analyses, and protein expression by Western blot analyses (FIG. 9).

### Immunoblot Analysis

Immunoblot analysis was done by resolving protein extracts from wild-type and transgenic plants on a uniform 12.5% SDS-PAGE, and then electrotransferring onto nitrocellulose membrane at 150 mA for 2 h. The membranes were blocked with 5% (w/v) non-fat milk for 1 h and incubated with anti-CaTLP1 polyclonal antibody for 2 h. The antibody was raised in rabbit against an antigenic peptide having amino acid sequence as set forth in SEQ ID NO: 3. (Sigma-Genosys, USA). The blot was eventually incubated with alkaline phosphatase conjugated secondary antibody for 1 h and the signals were detected using NBT/BCIP (nitro blue tetrazolium/5-bromo-4-chloro-3-indolyl phosphate) method (FIG. 9).

None of the transgenic lines displayed any phenotypic abnormality throughout their life cycle. Two independent overexpressing lines from F2 generation, one with high (T1) and the other with moderate expression (T4) of the transgene, were selected for further characterization. Southern blot analysis revealed the integration of three and two copies of the CaTLP1 in T1 and T4 lines, respectively (FIG. 9).

### Phenotypic Characteristics of Transgenic Plants

The CaTLP1-overexpressing tobacco seedlings were transplanted and grown alongside the wild-type plants under identical conditions. The transgenic plants displayed a higher growth rate, as evident by increased shoot height and biomass, when compared to wild-type plants (FIG. 10) Average root length in 15-d-old transgenic plants was more than two-fold than that of wild-type plants (FIG. 10). Similarly, the shoot length of the transgenic plants was significantly higher. The average area of leaves on the transgenic plants produced during early- to mid-vegetative phase (15 to 45-d-old plants) was about 2.5-fold higher than those of wild-type plants (FIG. 10). However, there was no appreciable change in the average area of leaves produced later in the vegetative phase and just prior to flowering. Since the average leaf area and primary root length of the transgenic plants were higher than those of wild-type plants, their anatomical features were examined and found to be significantly different. As shown in FIG. 10, the average area of epidermal cell of the leaves and epiblema cells of the roots of transgenic plants were 1.5-1.8 fold higher than those of wild-type plants, which is likely to be the main reason for their rapid growth.

### Stress Tolerance in Transgenic Plants

To determine the stress tolerance of the transgenic plants, chlorophyll retention rate was monitored by leaf-disc assay. Leaf-disc bioassay was carried out to determine dehydration, salinity, and oxidative stress tolerance using 1 cm² discs excised from 30-day-old, both wild-type and transgenic, tobacco plants.

Chlorosis was observed against osmotic or oxidative stress on the leaf-discs subjected to sorbitol (200 mM), NaCl (200 mM and 500 mM), H₂O₂ (2 mM) and menadione (5 μM and 7 μM) treatments. The discs floated on sterile distilled water served as experimental control. (b) Quantitation of chlorophyll content in leaf-discs of wild-type and transgenic plants under progressive osmotic stress of 250 mM NaCl.

For salinity experiments, the seedlings were supplemented with half-Hoagland's medium until three weeks, followed by treatment with 100, 250, and 500 mM of NaCl in the medium. The tissues were harvested 24 hours after the treatment.

Methyl jasmonate 100 μM was sprayed on three-week-old seedlings and tissues were collected at 8, 16, and 24 h for analysis.

Salicylic acid 5 mM solution was prepared in ethanol and sprayed on the leaflets of three-week-old seedlings. The harvested tissues were instantly frozen in liquid nitrogen and stored at −80° C., unless stated otherwise.

The chlorosis of the discs was physically examined after 72 h of incubation in the various stress inducer solutions. Chlorophyll retention assay was done in leaf-discs subjected to salinity stress as described earlier (Porra R J (2002). The chequered history of the development and use of simultaneous equations for the accurate determination of chlorophylls a and b. Photosynth Res 73: 149-156). The chlorophyll content was calculated using following formulas:

[Chl a]=(12.00 9*A_{663.8})−(3.11 9*A_{646.8}); (1)

[Chl b]=(20.78 9*A_{646.8})−(4.88 9*A_{663.8}); (2)

[Chl a+b]=(17.67 9*A_{646.8})+(7.12 9*A_{663.8}); (3)

where A is the absorption measured at respective wavelengths.

The leaf-discs of transgenic plants could retain higher percentage (30-45% as against the wild-type plants) of chlorophyll when compared with that of wild-type plants (FIG. 12).

### Evaluation of ABA-Mediated Stress Response

To evaluate ABA-mediated stress response, 8-day-old wild-type and transgenic seedlings were transferred to medium supplemented with 1 μM ABA and allowed to grow for 3 weeks. 100 μM ABA was sprayed on three-week-old seedlings and tissues were harvested at targeted intervals. The response was measured in terms of percent relative fresh weight, i.e. fresh weight of the seedlings grown in experimental conditions relative to the fresh weight of the same line grown in control conditions. The relative fresh weight was 58% in T1, followed by 50% in T4, and 31% in the wild-type plants (FIG. 12).

### Photosynthetic and Transpiration Rates

Net photosynthesis and rate of transpiration were measured using a gas exchange fluorescence system GFS3000 (Heinz Walz GmbH, Germany). The measurements were made under saturating irradiance (1000 μmol photons m⁻² s⁻¹) and constant airflow of 750 μl s⁻¹ at 25° C. Five independent leaves of three randomly chosen plants each, from wild-type and transgenic plants, were placed directly into the measuring cell (3 cm²) for the measurements. Each experiment was replicated at least three times and values are expressed as means±SE. All mean comparisons were done using paired t-test for independent samples. The measurements for determining net photosynthetic rate and leaf conductance were analyzed by one-way analysis of variance (ANOVA).

The three-week-old seedlings were subjected to dehydration condition for a period of seven days by withholding watering of the plants and tissues were harvested at different intervals of 24 hours. The pots containing dehydrated seedlings were then rewatered and allowed to recover for a period of 24 hours, and the rehydrated tissues (R24) were harvested.

Under progressive dehydration, the photosynthetic rate in transgenic plants remained unaltered in initial 24 hours, decreased linearly up to 96 h, and sustained thereafter. In contrast, the decrease in photosynthetic rate in wild-type plants was found to be proportional to the duration of stress treatment. In wild-type plants, photosynthetic rate reduced to 12% after 120 hours of dehydration, while the rates were 60% and 50% in transgenic T1 and T4 plants, respectively (FIG. 13). During the time-course of dehydration, the detached leaves of transgenic plants lost water more slowly, 15-20% less, as compared to wild-type plants (FIG. 13). Transgenic plants showed reduced rate of transpiration compared to wild-type plants subjected to dehydration (FIG. 13).

Forward Internal Primer Sequence for CaTLP1
SEQ ID NO: 1
TGGATAGTCAACCTCCACATG
Reverse Internal Primer Sequence for CaTLP1
SEQ ID NO: 2
GGTTGCCAGAGTATGAATCTG
Amino Acid Sequence for antigenic peptide
SEQ ID NO: 3
CVDPSHNVSSEEQER
cDNA Sequence for CaTLP1 gene
SEQ ID NO: 4
ATGCCATTGAAAAACATAGTCCGTGAATTCAAGGGGATGAAAAATGGAATAGGTAGTAAATCGAAGCGGGGTGG
TGACAGCAAGCATTGGCTTTGTCGGTCAAAGTCGCACGTTGCTCCGGATGTAACTCCATTTGAACCTATTCAAC
AGGGACAGTGGGCAAGTTTACCACCTGAATTGCTGTTGGACATAATCCGGAGGGTTGAAGAGAGTGAGACATCT
TGGCCTGCGCGCGCTGTTGTTGTTTTTTGTGCTTCGGTATGTAAATCATGGAGGTCGGTCACAAAGGAGATAAT
CAAGACCCCTCAGCAATGTGGAAGGATCACATTTCCTATTTCATTGAAGCAGCCCGGTCCCCGTGATTGTCCAA
TACAGTGCTTTATTAGGAGGAACAGGGAAAGTTCTACATTCTTGTTGTACTTAGGTTTGGTGCCATCGGAGCAT
GAGAGTAATAAGTTGTTATTAGTCGCCAGAAAGATAAGAAGGGCCACAGGCACCGAAATTGTCATATCTTTGGC
TGCAGACGATTTTTCTCGAGCAAGCAACAATTATGTTGGTAAACTGAGGTCTAACTTTTGGGGCACCAAGTTCA
CTATATACGATAGTCAACCTCCACATGATATTATAGTTCAACCAAATTGTCGAGGTGGGAAATTTAATTCTAAG
CAGGTTTCACCAAGAGCTCCAGCATGTAGCTATCTTGTAAGCTCCATTTCCTATGAGCTGAATACTCTCTGGAC
TAGAGGACCAAGAAGAATGCACTGCGTCATGAACTCTATACCCGTCTCAGCTATTGAGGAAGGAGGAAATGCTC
CAACTCCAACATTGTTACCTCAAATATTTGACGAACCTTTTTCTCCCTCACCGGCACTGAAAGGAAAAAGTCCA
ATGACAGATTCATACTCTGGCAACCTATCAGAGCTACCAGAACTAAGCCAAGGCTCGGTCGAGCCCTTGGTACT
CAAAAACAAGGCTCCAAGATGGCATGAGCAGCTCCAGTGTTGGTGTCTAAACTTTGTCGGCCGTGTTACGGTGG
CATCTGTAAAGAACTTTCAACTTGTAGCTGCTGTTGATCCGTCTCATAATGTTTCTTCTGAAGAGCAAGAAAGG
GTAATCTTGCAGTTTGGAAAGATCGGAAAAGATATATTCACCATGGACTACTCTTATCCACTCTCTGCCTTCCA
AGCCTTTGCCATCTGTTTGACAAGCTTTGATACTAAACCAGCCTGTGAATGA
Amino Acid Sequence for CaTLP1 protein
SEQ ID NO: 5
MPLKNIVREFKGMKNGIGSKSKRGGDSKHWLCRSKSHVAPDVTPFEPIQQGQWASLPPELLLDIIRR
VEESETSWPARAVVVFCASVCKSWRSVTKEIIKTPQQCGRITFPISLKQPGPRDCPIQCFIRRNRES
STFLLYLGLVPSEHESNKLLLVARKIRRATGTEIVISLAADDFSRASNNYVGKLRSNFWGTKFTIYD
SQPPHDIIVQPNCRGGKFNSKQVSPRAPACSYLVSSISYELNTLWTRGPRRMHCVMNSIPVSAIEEG
GNAPTPTLLPQIFDEPFSPSPALKGKSPMTDSYSGNLSELPELSQGSVEPLVLKNKAPRWHEQLQCW
CLNFVGRVTVASVKNFQLVAAVDPSHNVSSEEQERVILQFGKIGKDIFTMDYSYPLSAFQAFAICLT
SFDTKPACE
Nucleotide Sequence for Genomic DNA of CaTLP1
SEQ ID NO: 6
GCGATTCTGTATAATCGCGGTGGCGGTGAAGTAACCGATGGCTACAAACTCAAATAACACTGTCTCAGCTTCTA
CTTCTTTTTCTTGGTTTTTCAGATCCGTTTGCTATTGATCTCTTCTTTCTTCTTCTTCATCTTCCAGTTTTGGA
AATTTAGAAGCTTGCGTTTGGATTGGTGCAGTTTGAAGTTGCAGCGGAGTTTGTAAGGTGTGGAAACATGCCAT
TGAAAAACATAGTCCGTGAATTCAAGGGGATGAAAAATGGAATAGGTAGTAAATCGAAGCGGGGTGGTGACAGC
AAGCATTGGCTTTGTCGGTCAAAGTCGCACGTTGCTCCGGATGTAACTCCATTTGGTAATAAATTATTCACTTT
ATTTTTTGGTCGAATCCAAATTATTCTTAAACCTCGGGAGATTTATGTTTCTTATGTTCTCTTTCGTTATGAAT
AGAACCTATTCAACAGGGACAGTGGGCAAGTTTACCACCTGAATTGCTGTTGGACATAATCCGGAGGGTTGAAG
AGAGTGAGACATCTTGGCCTGCGCTTCATTGCTGAAAACTTCTATTTCTTGCATTCATGACTATGAGCGTTGAA
TTTTAAGTTACCTGCTCTAAATTTATGGGCTTTTTTCCGTTTATTGTCATTCAATTTCAGATAATGGGTCATTG
CAGTGTTGTTAAATAGCGGCTACAACATAACGGAATTTTGAACAAAACGCTATTGTTCCGCATTATGCTATTTA
GTTCAAAATAGTGTCTAATGGCAGCTATAGCTGTGTTATAGCACTATTGCATAGAGGAATTTGAACAAACTGTT
ATTTTTCACGATCTGCGATTGACAAACATTGGTTCATTGTCATTTTTTTTGTATCTTGTGATCTACGTATTTAA
GCAGTTTGTTTTGGTTTGGTCGCAATTGTATAGCGGAGCAGCGCTGTTGTTGTTTTTTGTGCTTCGGTATGTAA
ATCATGGAGGTCGGTCACAAAGGAGATAATCAAGACCCCTCAGCAATGTGGAAGGATCACATTTCCTATTTCAT
TGAAGCAGCCCGATTTTCATCCTGCCCGTTATAATCTTTACGACTTGATTTTTTACATTTTGCAGGTCTAACTT
TTGGGGCACCAAGTTCACTATATACGATAGTCAAGTCCCCGTGATTGTCCAATACAGTGCTTTATTAGGAGGAA
CAGGGAAAGTTCTACATTCTTGTTGTACTTAGGTTTGGTGCCATCGGAGCATGAGAGTAATAAGTTGTTATTAG
TCGCCAGAAAGATAAGAAGGGCCACAGGCACCGAAATTGTCATATCTTTGGCTGCAGACGATTTTTCTCGAGCA
AGCAACAATTATGTTGGTAAACTGAGGTCTAACTTTTGGGGCACCAAGTTCACTATATACGATAGTCAACCTCC
ACATGATATTATAGTTCAACCAAATTGTCGAGGTGGGAAATTTAATTCTAAGCAGGTTTCACCAAGAGCTCCAG
CATGTAGCTATCTTGTAAGCTCCATTTCCTATGAGCTGAATACTCTCTGGACTAGAGGACCAAGAAGAATGCAC
TGCGTCATGAACTCTATACCCGTCTCAGCTATTGAGGAAGGAGGAAATGCTCCAACTCCAACATTGTTACCTCA
AATATTTGACGAACCTTTTTCTCCCTCACCGGCACTGAAAGGAAAAAGTCCAATGACAGATTCATACTCTGGCA
ACCTATCAGAGCTACCAGAACTAAGCCAAGGCTCGGTCGAGCCCTTGGTACTCAAAAACAAGGCTCCAAGATGG
CATGAGCAGCTCCAGTGTTGGTGTCTAAACTTTGTCGGCCGTGTTACGGTGGCATCTGTAAAGAACTTTCAACT
TGTAGCTGCTGTTGATCCGTCTCATAATGTTTCTTCTGAAGAGCAAGAAAGGGTAATCTTGCAGTTTGGAAAGA
TCGGAAAAGATATATTCACCATGGACTACTCTTATCCACTCTCTGCCTTCCAAGCCTTTGCCATCTGTTTGACA
AGCTTTGATACTAAACCAGCCTGTGAATGAGAGCCGTGTTACTGCATTCGTGATCATCATCTGCTGATTTGTTG
CATGTAAAAATGCTAGCGCAGCCGCACTGTTCATCAAGCACTGAGCACCGTGGCATGCATGTGAAAATCTCAGC
TATGCAATCATTTGTATTCTCCCCCCTTGTATAAGTTGTATTGATTCTGGAATTTATTTTGTATGTTATTTCAT
AGTGTCCTCTGACTAAAATTAAATTAAACGATGTTTAATGAGTTTTATAATATATACATATTATTTGCTAAAAA
AAAAA
Nucleotide Sequence for full-length ORF of CaTLP1
SEQ ID NO: 7
GCGATTCTGTATAATCGCGGTGGCGGTGAAGTAACCGATGGCTACAAACTCAAATAACACTGTCTCAGCTTCTA
CTTCTTTTTCTTGGTTTTTCAGATCCGTTTGCTATTGATCTCTTCTTTCTTCTTCTTCATCTTCCAGTTTTGGA
AATTTAGAAGCTTGCGTTTGGATTGGTGCAGTTTGAAGTTGCAGCGGAGTTTGTAAGGTGTGGAAACATGCCAT
TGAAAAACATAGTCCGTGAATTCAAGGGGATGAAAAATGGAATAGGTAGTAAATCGAAGCGGGGTGGTGACAGC
AAGCATTGGCTTTGTCGGTCAAAGTCGCACGTTGCTCCGGATGTAACTCCATTTGAACCTATTCAACAGGGACA
GTGGGCAAGTTTACCACCTGAATTGCTGTTGGACATAATCCGGAGGGTTGAAGAGAGTGAGACATCTTGGCCTG
CGCGCGCTGTTGTTGTTTTTTGTGCTTCGGTATGTAAATCATGGAGGTCGGTCACAAAGGAGATAATCAAGACC
CCTCAGCAATGTGGAAGGATCACATTTCCTATTTCATTGAAGCAGCCCGGTCCCCGTGATTGTCCAATACAGTG
CTTTATTAGGAGGAACAGGGAAAGTTCTACATTCTTGTTGTACTTAGGTTTGGTGCCATCGGAGCATGAGAGTA
ATAAGTTGTTATTAGTCGCCAGAAAGATAAGAAGGGCCACAGGCACCGAAATTGTCATATCTTTGGCTGCAGAC
GATTTTTCTCGAGCAAGCAACAATTATGTTGGTAAACTGAGGTCTAACTTTTGGGGCACCAAGTTCACTATATA
CGATAGTCAACCTCCACATGATATTATAGTTCAACCAAATTGTCGAGGTGGGAAATTTAATTCTAAGCAGGTTT
CACCAAGAGCTCCAGCATGTAGCTATCTTGTAAGCTCCATTTCCTATGAGCTGAATACTCTCTGGACTAGAGGA
CCAAGAAGAATGCACTGCGTCATGAACTCTATACCCGTCTCAGCTATTGAGGAAGGAGGAAATGCTCCAACTCC
AACATTGTTACCTCAAATATTTGACGAACCTTTTTCTCCCTCACCGGCACTGAAAGGAAAAAGTCCAATGACAG
ATTCATACTCTGGCAACCTATCAGAGCTACCAGAACTAAGCCAAGGCTCGGTCGAGCCCTTGGTACTCAAAAAC
AAGGCTCCAAGATGGCATGAGCAGCTCCAGTGTTGGTGTCTAAACTTTGTCGGCCGTGTTACGGTGGCATCTGT
AAAGAACTTTCAACTTGTAGCTGCTGTTGATCCGTCTCATAATGTTTCTTCTGAAGAGCAAGAAAGGGTAATCT
TGCAGTTTGGAAAGATCGGAAAAGATATATTCACCATGGACTACTCTTATCCACTCTCTGCCTTCCAAGCCTTT
GCCATCTGTTTGACAAGCTTTGATACTAAACCAGCCTGTGAATGAGAGCCGTGTTACTGCATTCGTGATCATCA
TCTGCTGATTTGTTGCATGTAAAAATGCTAGCGCAGCCGCACTGTTCATCAAGCACTGAGCACCGTGGCATGCA
TGTGAAAATCTCAGCTATGCAATCATTTGTATTCTCCCCCCTTGTATAAGTTGTATTGATTCTGGAATTTATTT
TGTATGTTATTTCATAGTGTCCTCTGACTAAAATTAAATTAAACGATGTTTAATGAGTTTTATAATATATACAT
ATTATTTGCTAAAAAAAAAA
Nucleotide Sequence of 5′ UTR of CaTLP1
SEQ ID NO: 8
GCGATTCTGTATAATCGCGGTGGCGGTGAAGTAACCGATGGCTACAAACTCAAATAACACTGTCTCAGCTTCTA
CTTCTTTTTCTTGGTTTTTCAGATCCGTTTGCTATTGATCTCTTCTTTCTTCTTCTTCATCTTCCAGTTTTGGA
AATTTAGAAGCTTGCGTTTGGATTGGTGCAGTTTGAAGTTGCAGCGGAGTTTGTAAGGTGTGGAAAC
Nucleotide Sequence of 3′ UTR of CaTLP1
SEQ ID NO: 9
GAGCCGTGTTACTGCATTCGTGATCATCATCTGCTGATTTGTTGCATGTAAAAATGCTAGCGCAGCCGCACTGT
TCATCAAGCACTGAGCACCGTGGCATGCATGTGAAAATCTCAGCTATGCAATCATTTGTATTCTCCCCCCTTGT
ATAAGTTGTATTGATTCTGGAATTTATTTTGTATGTTATTTCATAGTGTCCTCTGACTAAAATTAAATTAAACG
ATGTTTAATGAGTTTTATAATATATACATATTATTTGCTAAAAAAAAAA
Forward Primer Sequence for amplification of CaTLP1 cDNA
SEQ ID NO: 10
CGGGATCCATGCCATTGAAAAACAT
Reverse Primer Sequence for amplification of CaTLP1 cDNA
SEQ ID NO: 11
GCTCTAGATCATTCACAGGCTGGTT

1. A polynucleotide encoding the protein having the amino acid sequence as set forth in SEQ ID NO: 5.

2. The polynucleotide as claimed in claim 1, wherein the nucleotide sequence of said polynucleotide has at least 90% sequence identity with the nucleotide sequence as set forth in SEQ ID NO: 4, SEQ ID NO: 6, or SEQ ID NO: 7.

3. The polynucleotide as claimed in claim 1, wherein the nucleotide sequence of said polynucleotide is as set forth in SEQ ID NO: 4, SEQ ID NO: 6, or SEQ ID NO: 7.

4. The polynucleotide as claimed in claim 1, wherein said polynucleotide is responsible for conferring enhanced tolerance to abiotic stress in a transformed plant as compared to an untransformed plant.

5. The polynucleotide as claimed in claim 4, wherein said abiotic stress is selected from the group consisting of dehydration stress, salinity stress, oxidative stress, osmotic stress, drought stress, and phytohormone stress.

6. The polynucleotide as claimed in claim 1, wherein said polynucleotide is responsible for enhanced plant growth and development in a transformed plant as compared to an untransformed plant.

7. A recombinant DNA construct comprising
a. the polynucleotide as claimed in claim 1; or
b. the polynucleotide having the nucleotide sequence as set forth in SEQ ID NO: 4; or
c. the polynucleotide having the nucleotide sequence as set forth in SEQ ID NO: 6; or
d. the polynucleotide having the nucleotide sequence as set forth in SEQ ID NO: 7
wherein said polynucleotide is operably linked to a promoter.

8. A recombinant vector comprising the recombinant DNA construct as claimed in claim 7.

9. A recombinant host cell comprising a polynucleotide encoding the protein having the amino acid sequence as set forth in SEQ ID NO: 5 or the recombinant vector as claimed in claim 8.

10. The recombinant host cell as claimed in claim 9, wherein said host cell is selected from the group consisting of E. coli, Agrobacterium, yeast and, plant cell.

11. A polypeptide having amino acid sequence as set forth in SEQ ID NO: 5, wherein expression of said polypeptide is responsible for conferring enhanced tolerance to abiotic stress in a transformed plant as compared to an untransformed plant.

12. A method for producing a transformed plant cell, plant, or a part thereof over-expressing a protein having the amino acid sequence as set forth in SEQ ID NO: 5, wherein said method comprises:
(a) transforming said plant cell, plant, or part thereof with a polynucleotide encoding said protein having the amino acid sequence as set forth in SEQ ID NO: 5, and
(b) selecting the transformed plant cell, plant or part thereof,
wherein said transformed plant cell, plant, or part thereof is more tolerant to abiotic stress than an untransformed plant.

13. The method as claimed in claim 12, wherein said polynucleotide is as set forth in SEQ ID NO: 4.

14. The method as claimed in claim 12, wherein said method further comprises regenerating the transformed plant cell, plant, or part thereof into a transformed plant, wherein said transformed plant is more tolerant to abiotic stress than an untransformed plant, and is morphologically similar to the untransformed plant.

15. The method as claimed in claim 12, wherein said abiotic stress is selected from the group consisting of dehydration stress, salinity stress, oxidative stress, osmotic stress, drought stress, and phytohormone stress.

16. The method as claimed in claim 12, wherein the plant is a monocotyledonous or dicotyledonous plant.

17. The method as claimed in claim 12, wherein the plant is selected from the group consisting of tobacco, potato, sweet potato, cassava, sugar-beet, Arabidopsis, brassica species, tomato, brinjal, capsicum, chilli, okra, cucurbit, melon, mulberry, banana, mango, papaya, alfalfa, grass, canola, sunflower, cotton, legume plant, groundnut, peanut, pea, soybean, chickpea, pigeon pea, mungbean, medicago, lotus, Petunia, rice, maize, wheat, rye, barley, oats, pearl millet, corn, sorghum, nuts, avocado, turmeric, saffron, ginger, garlic, onion, nutmeg, forage plant, fruit tree, and ornamental plant.

18. The method as claimed in claim 12, wherein plant is transformed using a method selected from the group consisting of floral dip method, Agrobacterium mediated transformation method, protoplast mediated transformation, particle gun bombardment method, in planta transformation, and liposome mediated transformation method.

19. A transgenic plant, plant cell or progeny thereof over-expressing a protein having the amino acid sequence as set forth in SEQ ID NO: 5, wherein the transgenic plant, plant cell or progeny thereof is more tolerant to abiotic stress than an untransformed plant.

20. The transgenic plant, plant cell or progeny thereof as claimed in claim 17, wherein the transgenic plant comprises the polynucleotide sequence as set forth in SEQ ID NO: 4 or the recombinant DNA construct comprising:
a. a polynucleotide encoding the protein having the amino acid sequence as set forth in SEQ ID NO: 5; or
b. the polynucleotide having the nucleotide sequence as set forth in SEQ ID NO: 4; or
c. the polynucleotide having the nucleotide sequence as set forth in SEQ ID NO: 6, or
d. the polynucleotide having the nucleotide sequence as set forth in SEQ ID NO: 7;
wherein said polynucleotide is operably linked to a promoter.

21. The transgenic plant, plant cell or progeny thereof as claimed in claim 17, wherein said transgenic plant is selected from a group consisting of tobacco, potato, sweet potato, cassava, sugar-beet, Arabidopsis, Brassica species, tomato, brinjal, capsicum, chilli, okra, cucurbit, melon, mulberry, banana, mango, vitis, papaya, alfalfa, grass, canola, sunflower, cotton, legume plant, groundnut, peanut, pea, soybean, chickpea, pigeon pea, mungbean, medicago, lotus, petunia, rice, maize, wheat, rye, barley, oats, pearl millet, corn, sorghum, nuts, avocado, turmeric, saffron, ginger, garlic, onion, nutmeg, forage plant, fruit tree and ornamental plant.


SEQUENCE LISTING (11 sequences)

SEQ ID NO: 1 - DNA, 21 bp, ARTIFICIAL SEQUENCE
  Forward Internal Primer Sequence for CaTLP1
tggatagtcaacctccacatg

SEQ ID NO: 2 - DNA, 21 bp, ARTIFICIAL SEQUENCE
  Reverse Internal Primer Sequence for CaTLP1
ggttgccagagtatgaatctg

SEQ ID NO: 3 - protein, 15 aa, Cicer arietinum
CVDPSHNVSSEEQER

SEQ ID NO: 4 - DNA, 1236 bp, ARTIFICIAL SEQUENCE
  cDNA  Sequence for CaTLP1 gene
atgccattgaaaaacatagtccgtgaattcaaggggatgaaaaatggaataggtagtaaatcgaagcggggtggtgacagcaagcattggctttgtcggtcaaagtcgcacgttgctccggatgtaactccatttgaacctattcaacagggacagtgggcaagtttaccacctgaattgctgttggacataatccggagggttgaagagagtgagacatcttggcctgcgcgcgctgttgttgttttttgtgcttcggtatgtaaatcatggaggtcggtcacaaaggagataatcaagacccctcagcaatgtggaaggatcacatttcctatttcattgaagcagcccggtccccgtgattgtccaatacagtgctttattaggaggaacagggaaagttctacattcttgttgtacttaggtttggtgccatcggagcatgagagtaataagttgttattagtcgccagaaagataagaagggccacaggcaccgaaattgtcatatctttggctgcagacgatttttctcgagcaagcaacaattatgttggtaaactgaggtctaacttttggggcaccaagttcactatatacgatagtcaacctccacatgatattatagttcaaccaaattgtcgaggtgggaaatttaattctaagcaggtttcaccaagagctccagcatgtagctatcttgtaagctccatttcctatgagctgaatactctctggactagaggaccaagaagaatgcactgcgtcatgaactctatacccgtctcagctattgaggaaggaggaaatgctccaactccaacattgttacctcaaatatttgacgaacctttttctccctcaccggcactgaaaggaaaaagtccaatgacagattcatactctggcaacctatcagagctaccagaactaagccaaggctcggtcgagcccttggtactcaaaaacaaggctccaagatggcatgagcagctccagtgttggtgtctaaactttgtcggccgtgttacggtggcatctgtaaagaactttcaacttgtagctgctgttgatccgtctcataatgtttcttctgaagagcaagaaagggtaatcttgcagtttggaaagatcggaaaagatatattcaccatggactactcttatccactctctgccttccaagcctttgccatctgtttgacaagctttgatactaaaccagcctgtgaatga

SEQ ID NO: 5 - protein, 411 aa, Cicer arietinum
MPLKNIVREFKGMKNGIGSKSKRGGDSKHWLCRSKSHVAPDVTPFEPIQQGQWASLPPELLLDIIRRVEESETSWPARAVVVFCASVCKSWRSVTKEIIKTPQQCGRITFPISLKQPGPRDCPIQCFIRRNRESSTFLLYLGLVPSEHESNKLLLVARKIRRATGTEIVISLAADDFSRASNNYVGKLRSNFWGTKFTIYDSQPPHDIIVQPNCRGGKFNSKQVSPRAPACSYLVSSISYELNTLWTRGPRRMHCVMNSIPVSAIEEGGNAPTPTLLPQIFDEPFSPSPALKGKSPMTDSYSGNLSELPELSQGSVEPLVLKNKAPRWHEQLQCWCLNFVGRVTVASVKNFQLVAAVDPSHNVSSEEQERVILQFGKIGKDIFTMDYSYPLSAFQAFAICLTSFDTKPACE

SEQ ID NO: 6 - DNA, 2299 bp, Cicer arietinum
gcgattctgtataatcgcggtggcggtgaagtaaccgatggctacaaactcaaataacactgtctcagcttctacttctttttcttggtttttcagatccgtttgctattgatctcttctttcttcttcttcatcttccagttttggaaatttagaagcttgcgtttggattggtgcagtttgaagttgcagcggagtttgtaaggtgtggaaacatgccattgaaaaacatagtccgtgaattcaaggggatgaaaaatggaataggtagtaaatcgaagcggggtggtgacagcaagcattggctttgtcggtcaaagtcgcacgttgctccggatgtaactccatttggtaataaattattcactttattttttggtcgaatccaaattattcttaaacctcgggagatttatgtttcttatgttctctttcgttatgaatagaacctattcaacagggacagtgggcaagtttaccacctgaattgctgttggacataatccggagggttgaagagagtgagacatcttggcctgcgcttcattgctgaaaacttctatttcttgcattcatgactatgagcgttgaattttaagttacctgctctaaatttatgggcttttttccgtttattgtcattcaatttcagataatgggtcattgcagtgttgttaaatagcggctacaacataacggaattttgaacaaaacgctattgttccgcattatgctatttagttcaaaatagtgtctaatggcagctatagctgtgttatagcactattgcatagaggaatttgaacaaactgttatttttcacgatctgcgattgacaaacattggttcattgtcattttttttgtatcttgtgatctacgtatttaagcagtttgttttggtttggtcgcaattgtatagcggagcagcgctgttgttgttttttgtgcttcggtatgtaaatcatggaggtcggtcacaaaggagataatcaagacccctcagcaatgtggaaggatcacatttcctatttcattgaagcagcccgattttcatcctgcccgttataatctttacgacttgattttttacattttgcaggtctaacttttggggcaccaagttcactatatacgatagtcaagtccccgtgattgtccaatacagtgctttattaggaggaacagggaaagttctacattcttgttgtacttaggtttggtgccatcggagcatgagagtaataagttgttattagtcgccagaaagataagaagggccacaggcaccgaaattgtcatatctttggctgcagacgatttttctcgagcaagcaacaattatgttggtaaactgaggtctaacttttggggcaccaagttcactatatacgatagtcaacctccacatgatattatagttcaaccaaattgtcgaggtgggaaatttaattctaagcaggtttcaccaagagctccagcatgtagctatcttgtaagctccatttcctatgagctgaatactctctggactagaggaccaagaagaatgcactgcgtcatgaactctatacccgtctcagctattgaggaaggaggaaatgctccaactccaacattgttacctcaaatatttgacgaacctttttctccctcaccggcactgaaaggaaaaagtccaatgacagattcatactctggcaacctatcagagctaccagaactaagccaaggctcggtcgagcccttggtactcaaaaacaaggctccaagatggcatgagcagctccagtgttggtgtctaaactttgtcggccgtgttacggtggcatctgtaaagaactttcaacttgtagctgctgttgatccgtctcataatgtttcttctgaagagcaagaaagggtaatcttgcagtttggaaagatcggaaaagatatattcaccatggactactcttatccactctctgccttccaagcctttgccatctgtttgacaagctttgatactaaaccagcctgtgaatgagagccgtgttactgcattcgtgatcatcatctgctgatttgttgcatgtaaaaatgctagcgcagccgcactgttcatcaagcactgagcaccgtggcatgcatgtgaaaatctcagctatgcaatcatttgtattctccccccttgtataagttgtattgattctggaatttattttgtatgttatttcatagtgtcctctgactaaaattaaattaaacgatgtttaatgagttttataatatatacatattatttgctaaaaaaaaaa

SEQ ID NO: 7 - DNA, 1722 bp, Cicer arietinum
gcgattctgtataatcgcggtggcggtgaagtaaccgatggctacaaactcaaataacactgtctcagcttctacttctttttcttggtttttcagatccgtttgctattgatctcttctttcttcttcttcatcttccagttttggaaatttagaagcttgcgtttggattggtgcagtttgaagttgcagcggagtttgtaaggtgtggaaacatgccattgaaaaacatagtccgtgaattcaaggggatgaaaaatggaataggtagtaaatcgaagcggggtggtgacagcaagcattggctttgtcggtcaaagtcgcacgttgctccggatgtaactccatttgaacctattcaacagggacagtgggcaagtttaccacctgaattgctgttggacataatccggagggttgaagagagtgagacatcttggcctgcgcgcgctgttgttgttttttgtgcttcggtatgtaaatcatggaggtcggtcacaaaggagataatcaagacccctcagcaatgtggaaggatcacatttcctatttcattgaagcagcccggtccccgtgattgtccaatacagtgctttattaggaggaacagggaaagttctacattcttgttgtacttaggtttggtgccatcggagcatgagagtaataagttgttattagtcgccagaaagataagaagggccacaggcaccgaaattgtcatatctttggctgcagacgatttttctcgagcaagcaacaattatgttggtaaactgaggtctaacttttggggcaccaagttcactatatacgatagtcaacctccacatgatattatagttcaaccaaattgtcgaggtgggaaatttaattctaagcaggtttcaccaagagctccagcatgtagctatcttgtaagctccatttcctatgagctgaatactctctggactagaggaccaagaagaatgcactgcgtcatgaactctatacccgtctcagctattgaggaaggaggaaatgctccaactccaacattgttacctcaaatatttgacgaacctttttctccctcaccggcactgaaaggaaaaagtccaatgacagattcatactctggcaacctatcagagctaccagaactaagccaaggctcggtcgagcccttggtactcaaaaacaaggctccaagatggcatgagcagctccagtgttggtgtctaaactttgtcggccgtgttacggtggcatctgtaaagaactttcaacttgtagctgctgttgatccgtctcataatgtttcttctgaagagcaagaaagggtaatcttgcagtttggaaagatcggaaaagatatattcaccatggactactcttatccactctctgccttccaagcctttgccatctgtttgacaagctttgatactaaaccagcctgtgaatgagagccgtgttactgcattcgtgatcatcatctgctgatttgttgcatgtaaaaatgctagcgcagccgcactgttcatcaagcactgagcaccgtggcatgcatgtgaaaatctcagctatgcaatcatttgtattctccccccttgtataagttgtattgattctggaatttattttgtatgttatttcatagtgtcctctgactaaaattaaattaaacgatgtttaatgagttttataatatatacatattatttgctaaaaaaaaaa

SEQ ID NO: 8 - DNA, 215 bp, Cicer arietinum
gcgattctgtataatcgcggtggcggtgaagtaaccgatggctacaaactcaaataacactgtctcagcttctacttctttttcttggtttttcagatccgtttgctattgatctcttctttcttcttcttcatcttccagttttggaaatttagaagcttgcgtttggattggtgcagtttgaagttgcagcggagtttgtaaggtgtggaaac

SEQ ID NO: 9 - DNA, 271 bp, Cicer arietinum
gagccgtgttactgcattcgtgatcatcatctgctgatttgttgcatgtaaaaatgctagcgcagccgcactgttcatcaagcactgagcaccgtggcatgcatgtgaaaatctcagctatgcaatcatttgtattctccccccttgtataagttgtattgattctggaatttattttgtatgttatttcatagtgtcctctgactaaaattaaattaaacgatgtttaatgagttttataatatatacatattatttgctaaaaaaaaaa

SEQ ID NO: 10 - DNA, 25 bp, ARTIFICIAL SEQUENCE
  Forward Primer Sequence for amplification of CaTLP1 cDNA
cgggatccatgccattgaaaaacat

SEQ ID NO: 11 - DNA, 25 bp, ARTIFICIAL SEQUENCE
  Reverse Primer Sequence for amplification of CaTLP1 cDNA
gctctagatcattcacaggctggtt